SEQUENCE LISTING (21 sequences)

SEQ ID NO: 1 - DNA, 2167 bp, Oryza sativa
cgcggatccgaacactgcgtttgctggctttgatgaaagtgtccagcatttccatcttctcattgtgtcgatactctcactgatgtgtggtttttcatgacatcttaataggaaaacgaggagatgagcaaagtaactcaagctaatggaatagactccagggaacacaaagggcagagcatagcagttgaagccaacagcaaaggagagactcgtgaagtcgttgaagggaaggttgactggaggggaagacctgcaataagagggagtcatggtggtgttgccaattctttcttcattcttgtgaactttgggctcgagaaccttgcctcgttgtctctagccgtgaaccttatcatctacttcatgacggtcatgcacattggtctgactgatggctccaacctgctgaccaactacatgggcacgagctacatggtcgcagtgctcatctccgtcttcgcggacactttcattggccggtacaagactgtcatcatatcgtcagtgatcgagctcgtgggcctgctgattcttacactgcaagctcactccaataagctgaagccaccgtattgcgtcttcccgttcgaccccaagtgcgagacggtgagcggcgacggcaggacgcacctctacgtggggctgtacctcgtggcgatcggctcggcgggcatcaaggcggcgctgccggcgcactgcgccgaccagttcgacgagaagcaccccacggagaagctgcagatgtccagcttcttcaactggctgctgctcagcctctgcaccggcggcgccatcagcgtcacggtgttcgtgtggatccagagctacaagggctgggacaaagggttcggcgccgccaccggcgtgatgggcctcgccctcctcgtcttcatcgccggcctccccgggtaccgcatctccgtcgtgcagggcagcaccgcgcttcttgaaatcttgcaggtgtatgttgctgccatcaggaacaggaatatgaagctccctgagaacccagacgagctgtacgagatcagcaagagcaaagctccccctgacacggacttcatggctcacagggataaaccgttcaggttccttgacaaggcggcgatagtacaggcgccaacggatgaggcgccgagcccatggcggcagtgccgagtgacccaggtggagcacgccaagacggtgctcgccatggtgcccatcttctgcagcgccatcatcatgagcacctgcctcgcgcagctccagacattctccatccagcagggcgtcaccatggacaggaccatcggcacgttcaagatgccgccggcgtcgctgcccatcatcccgctcatcgtcctcgtgttcgcggtgcccatctacgagcggggcttcgtgcccttcgcccgccgcatcaccggccaccccaacggcatcccgcacctgcagcgggtcggcgtcggcctcgtgctctccatcgtctccatggccatcgccgccgtcgtggaggtgcgccgcaagagggtggcagcaaggcacgggatgctggacgcgaatcccattctcgggaagcagctgcccatctcctgcttctggctggcgccgcagttcaccgtgttcggcgtcgctgacatgttcaccttcatcgggctcctcgagttcttctactcgcaggcgccgccggcgctcaagtccatgtcatcctcgttcctgtggtgccccatgtcgctcgggtacttcctcagcaccatcatcgtcaaggctgtgaacgccgccaccaggggcgccacggcgagcggcggctggttggccggcaacaacatcaaccggaaccacctcgacctcttcttctggctgctcgccgtgctcagcttcctcaacttcctcaactacctcttctgggccagctggtacaagtacaagcctcagcagtcagcccacgtaccagcagagcacaaagtatgatgatcatccagcgtctcgagaaccttggtaataaaagatgtgcagttgttgctaccatttttgcctcacgggtgcacggagtaccaacgtggcaacgtgagtttcccataaggggaagaatatataggtatacggaacttaattctaatcagttccagtacaaagatcatggaattgcatcgtcggtgtgtataaatgtgatattttgaagcac

SEQ ID NO: 2 - protein, 609 aa, Oryza sativa
MSKVTQANGIDSREHKGQSIAVEANSKGETREVVEGKVDWRGRPAIRGSHGGVANSFFILVNFGLENLASLSLAVNLIIYFMTVMHIGLTDGSNLLTNYMGTSYMVAVLISVFADTFIGRYKTVIISSVIELVGLLILTLQAHSNKLKPPYCVFPFDPKCETVSGDGRTHLYVGLYLVAIGSAGIKAALPAHCADQFDEKHPTEKLQMSSFFNWLLLSLCTGGAISVTVFVWIQSYKGWDKGFGAATGVMGLALLVFIAGLPGYRISVVQGSTALLEILQVYVAAIRNRNMKLPENPDELYEISKSKAPPDTDFMAHRDKPFRFLDKAAIVQAPTDEAPSPWRQCRVTQVEHAKTVLAMVPIFCSAIIMSTCLAQLQTFSIQQGVTMDRTIGTFKMPPASLPIIPLIVLVFAVPIYERGFVPFARRITGHPNGIPHLQRVGVGLVLSIVSMAIAAVVEVRRKRVAARHGMLDANPILGKQLPISCFWLAPQFTVFGVADMFTFIGLLEFFYSQAPPALKSMSSSFLWCPMSLGYFLSTIIVKAVNAATRGATASGGWLAGNNINRNHLDLFFWLLAVLSFLNFLNYLFWASWYKYKPQQSAHVPAEHKV

SEQ ID NO: 3 - DNA, 1830 bp, Oryza sativa
atgagcaaagtaactcaagctaatggaatagactccagggaacacaaagggcagagcatagcagttgaagccaacagcaaaggagagactcgtgaagtcgttgaagggaaggttgactggaggggaagacctgcaataagagggagtcatggtggtgttgccaattctttcttcattcttgtgaactttgggctcgagaaccttgcctcgttgtctctagccgtgaaccttatcatctacttcatgacggtcatgcacattggtctgactgatggctccaacctgctgaccaactacatgggcacgagctacatggtcgcagtgctcatctccgtcttcgcggacactttcattggccggtacaagactgtcatcatatcgtcagtgatcgagctcgtgggcctgctgattcttacactgcaagctcactccaataagctgaagccaccgtattgcgtcttcccgttcgaccccaagtgcgagacggtgagcggcgacggcaggacgcacctctacgtggggctgtacctcgtggcgatcggctcggcgggcatcaaggcggcgctgccggcgcactgcgccgaccagttcgacgagaagcaccccacggagaagctgcagatgtccagcttcttcaactggctgctgctcagcctctgcaccggcggcgccatcagcgtcacggtgttcgtgtggatccagagctacaagggctgggacaaagggttcggcgccgccaccggcgtgatgggcctcgccctcctcgtcttcatcgccggcctccccgggtaccgcatctccgtcgtgcagggcagcaccgcgcttcttgaaatcttgcaggtgtatgttgctgccatcaggaacaggaatatgaagctccctgagaacccagacgagctgtacgagatcagcaagagcaaagctccccctgacacggacttcatggctcacagggataaaccgttcaggttccttgacaaggcggcgatagtacaggcgccaacggatgaggcgccgagcccatggcggcagtgccgagtgacccaggtggagcacgccaagacggtgctcgccatggtgcccatcttctgcagcgccatcatcatgagcacctgcctcgcgcagctccagacattctccatccagcagggcgtcaccatggacaggaccatcggcacgttcaagatgccgccggcgtcgctgcccatcatcccgctcatcgtcctcgtgttcgcggtgcccatctacgagcggggcttcgtgcccttcgcccgccgcatcaccggccaccccaacggcatcccgcacctgcagcgggtcggcgtcggcctcgtgctctccatcgtctccatggccatcgccgccgtcgtggaggtgcgccgcaagagggtggcagcaaggcacgggatgctggacgcgaatcccattctcgggaagcagctgcccatctcctgcttctggctggcgccgcagttcaccgtgttcggcgtcgctgacatgttcaccttcatcgggctcctcgagttcttctactcgcaggcgccgccggcgctcaagtccatgtcatcctcgttcctgtggtgccccatgtcgctcgggtacttcctcagcaccatcatcgtcaaggctgtgaacgccgccaccaggggcgccacggcgagcggcggctggttggccggcaacaacatcaaccggaaccacctcgacctcttcttctggctgctcgccgtgctcagcttcctcaacttcctcaactacctcttctgggccagctggtacaagtacaagcctcagcagtcagcccacgtaccagcagagcacaaagtatga

SEQ ID NO: 4 - DNA, 20 bp, Artificial Sequence
  OsACTIN QF Primer
caacacccctgctatgtacg

SEQ ID NO: 5 - DNA, 21 bp, Artificial Sequence
  OsACTIN QR Primer
catcaccagagtccaacacaa

SEQ ID NO: 6 - DNA, 24 bp, Artificial Sequence
  OsNPF4.5 QF Primer
cgccgtgctcagcttcctcaactt

SEQ ID NO: 7 - DNA, 24 bp, Artificial Sequence
  OsNPF4.5 QR Primer
aggcaaaaatggtagcaacaactg

SEQ ID NO: 8 - DNA, 31 bp, Artificial Sequence
  OsNPF4.5 OF Expression Vector
cgcgtcgacatgagcaaagtaactcaagcta

SEQ ID NO: 9 - DNA, 29 bp, Artificial Sequence
  OsNPF4.5 OR Expression Vector
gccaagctttcatactttgtgctctgctg

SEQ ID NO: 10 - DNA, 23 bp, Artificial Sequence
  OsNPF4.5 cDNA 5′ Outer Primer
ggccaatgaaagtgtccgcgaag

SEQ ID NO: 11 - DNA, 24 bp, Artificial Sequence
  OsNPF4.5 cDNA 5′ Inner Primer
acggctagagacaacgaggcaagg

SEQ ID NO: 12 - DNA, 18 bp, Artificial Sequence
  OsNPF4.5 cDNA 3′ Outer Primer
gccgcagttcaccgtgtt

SEQ ID NO: 13 - DNA, 19 bp, Artificial Sequence
  OsNPF4.5 cDNA 3′ Inner Primer
tcatcgggctcctcgagtt

SEQ ID NO: 14 - DNA, 20 bp, Artificial Sequence
  spacer1
ggggaagacctgcaataaga

SEQ ID NO: 15 - DNA, 20 bp, Artificial Sequence
  spacer2
gttcgaccccaagtgcgaga

SEQ ID NO: 16 - DNA, 20 bp, Artificial Sequence
  spacer3
gtgtggatccagagctacaa

SEQ ID NO: 17 - DNA, 26 bp, Artificial Sequence
  Partial Wild Type Premutation
gttcgaccccaagtgcgagacggtga

SEQ ID NO: 18 - DNA, 26 bp, Artificial Sequence
  Osnpf4.5-1
gttcgaccccaagtgcgtagacggtg

SEQ ID NO: 19 - DNA, 26 bp, Artificial Sequence
  Osnpf4.5-2
gttcgaccccaagtgcagacggtgag

SEQ ID NO: 20 - DNA, 26 bp, Artificial Sequence
  Partial Wild Type Premutation
gtgtggatccagagctacaagggctg

SEQ ID NO: 21 - DNA, 26 bp, Artificial Sequence
  Osnpf4.5-3
gtgtggatccagagctcaagggctgg

(19) United States
(12) Patent Application Publication
(10) Pub. No.: US 2023/0045368 A1
(43) Pub. Date: Feb. 9, 2023

(54) UTILIZATION OF NITRATE TRANSPORT PROTEINS TO ENHANCE PLANT GROWTH

(71) Applicants: Texas Tech University System, Lubbock, TX (US); Nanjing Agricultural University, Nanjing (CN)

(72) Inventors: Aiqun Chen, Nanjing (CN); Shuangshuang Wang, Nanjing (CN); Guohua XU, Nanjing (CN); Luis R. Herrera-Estrella, Lubbock, TX (US); Damar L. Lopez-Arredondo, Lubbock, TX (US)

(21) Appl. No.: 17/790,423

(22) PCT Filed: Jan. 4, 2021
(86) PCT No.: PCT/US2021/012056

§ 371 Date: Jun. 30, 2022

(30) Foreign Application Priority Data
Jan. 2, 2020 (CN) 202010000479.4

(51) Int. Cl. C07K 14/415; C12N 15/82

(52) U.S. Cl. CPC C07K 14/415; C12N 15/8243

(57) ABSTRACT

In some embodiments, the present disclosure pertains to a nitrate transporter gene that includes the nucleotide sequence shown in SEQ ID NO: 1 or a functional variant thereof having at least 65% sequence identity to SEQ ID NO: 1. In some embodiments, the present disclosure pertains to a nitrate transporter protein that includes the amino acid sequence shown in SEQ ID NO: 2 or a functional variant thereof having at least 65% sequence identity to SEQ ID NO: 2. In some embodiments, the present disclosure pertains to a method of enhancing growth in a plant by introducing a nitrate transporter gene of the present disclosure into the plant to result in the expression of the nitrate transporter protein in the plant. In some embodiments, the present disclosure pertains to genetically modified plants and recombinant expression vectors that include the nitrate transporter genes of the present disclosure.

### CROSS-REFERENCE TO RELATED APPLICATIONS

This application claims priority to CN Patent Application No. 202010000479.4, filed on Jan. 2, 2020. The entirety of the aforementioned application is incorporated herein by reference.

### BACKGROUND

Low availability of nitrogen is often a major limiting factor to crop yields in most nutrient-poor soils. As such, a need exists for more effective absorption and utilization of nitrogen nutrients in plants. Various embodiments of the present disclosure address the aforementioned need.

### SUMMARY

In some embodiments, the present disclosure pertains to a nitrate transporter gene. In some embodiments, the gene includes the nucleotide sequence shown in SEQ ID NO: 1 or a functional variant thereof. In some embodiments, the functional variant has at least 65% sequence identity to SEQ ID NO: 1.

In some embodiments, the present disclosure pertains to a nitrate transporter protein. In some embodiments, the protein includes the amino acid sequence shown in SEQ ID NO: 2 or a functional variant thereof. In some embodiments, the functional variant has at least 65% sequence identity to SEQ ID NO: 2.

In some embodiments, the present disclosure pertains to a method of enhancing plant growth. In some embodiments, the methods of the present disclosure include a step of introducing a nitrate transporter gene of the present disclosure into the plant. In some embodiments, the introducing results in the expression of a nitrate transporter protein of the present disclosure in the plant. In some embodiments, the expressed nitrate transporter protein enhances plant growth by enhancing nitrogen transport in the plant. In some embodiments, the methods of the present disclosure also include a step of associating the plant with an arbuscular mycorrhizal fungi.

In some embodiments, the present disclosure pertains to a genetically modified plant. In some embodiments, the genetically modified plant includes an introduced nitrate transporter gene of the present disclosure.

In some embodiments, the present disclosure pertains to a recombinant expression vector. In some embodiments, the recombinant expression vector includes a nitrate transporter gene of the present disclosure.

### DESCRIPTION OF THE DRAWINGS

FIG. 1 illustrates methods of enhancing nitrogen transport in a plant according to various aspects of the present disclosure.

FIG. 2 illustrates OsNPF4.5 intron/exon structure analysis (black boxes are exons). The numbers below the black box and the numbers above the lines represent different exons and introns, respectively.

FIGS. 3A and 3B illustrate that an overexpression of OsNPF4.5 in rice promotes rice growth (FIG. 3A) and nitrogen uptake (FIG. 3B). Legend: WT: wild type; and OX-2, OX-3, and OX-4 are three overexpressing transgenic lines.

FIG. 4 illustrates expression analysis of OsNPF4.5 in inoculated/uninoculated arbuscular mycorrhiza (AM) fungal rice plants. Legend: R: roots; L: leaves; and AMF: AM fungi (R. irregularis).

FIG. 5 illustrates a frog egg heterologous system that confirms OsNPF4.5 has nitrate transport activity. Legend: H₂O: negative control; and CHL1: a known nitrate in Arabidopsis as a positive control.

FIGS. 6A and 6B illustrate maps of binary expression vectors used to construct overexpression.

FIG. 7 illustrates identification of overexpression effects of rice transgenic lines OsNPF4.5. Legend: WT: wild type; and OX-[1-5] are five overexpressing transgenic lines.

FIGS. 8A and 8B illustrate that the overexpression of OsNPF4.5 in rice promotes nitrate uptake by rice roots. Legend: WT: wild type; and OX[1-5] are five overexpressing transgenic lines.

FIG. 9 illustrates sequencing verification to obtain three homozygous mutants of osnpf4.5: osnpf4.5-1, osnpf4.5-2, and osnpf4.5-3 (arrows mark the positions of base insertions or deletions).

FIGS. 10A and 10B illustrate that mutation of OsNPF4.5 reduces aboveground biomass (FIG. 10A) and nitrogen concentration (FIG. 10B) in rice. Legend WT: wild type; and osnpf4.5-1, osnpf4.5-2, and osnpf4.5-3 are three mutant materials.

FIGS. 11A-11D illustrate RNA sequencing analysis of the rice mycorrhizal and nonmycorrhizal roots. FIG. 11A illustrates a Venn diagram showing the relationships between genes that show statistically significant differential expression in response to AM symbiosis in roots. The up-regulated genes are shown in red color, while the down-regulated genes are indicated in yellow color. The genes with no significant alteration in transcripts are shown in the intersection.

FIG. 11B illustrates the 30 most significantly enriched pathways analyzed by Kyoto Encyclopedia of Genes and Genomes (KEGG) algorithm. FIG. 11C illustrates a heat map of the up-regulated genes involved in nitrogen transport and metabolism, as well as several previously described AM-up-regulated genes that were shown as marker genes. FIG. 11D illustrates quantitative (reverse transcription-polymerase chain reaction (RT-PCR) analysis showed a more than 500-fold upregulation of OsNPF4.5, and a 11-fold upregulation of OsAMT3.1, in response to AM symbiosis. The AM-specific Pi transporter gene OsPT11 and H⁺-ATPase gene OsHA1 were used as control genes. The relative expression level of the assayed genes was normalized to a constitutive Actin gene. Values are the means±standard error (S.E) of 3 biological replicates (n=3). The asterisks indicate significant differences, *P<0.05; **P<0.01, ***P<0.001.

FIGS. 12A-H illustrate that AM fungal colonization promotes rice growth and nitrate uptake. FIG. 12A illustrates a diagrammatic representation (not to scale) of the compartmented culture system used in the experiment. Two inoculated or mock-inoculated seedlings of wild type (WT) or mutant plants were grown in the middle root/fungal compartment (RFC), and watered weekly with a nutrient solution containing 2.5 mM NO₃⁻. The hyphal compartments (HCs) aside were watered with a nutrient solution containing equal amount of ¹⁵NO₃⁻. FIG. 12B illustrates biomass of inoculated and mock-inoculated plants. FIG. 12C illustrates an assay of ¹⁵N content in both roots and shoots of inoculated and mock-inoculated plants. FIGS. 12D-E illustrate N content of inoculated and mock-inoculated plants. FIGS. 12F-G illustrate P content of inoculated and mock-inoculated plants. FIG. 12H illustrates the percentage of N and P transferred via the mycorrhizal pathway. Values are the means±S.E of 5 independent biological replicates (n=5). The asterisks indicate significant differences, *P<0.05; **P<0.01, ***P<0.001.

FIGS. 13A-H illustrate tissue-specific expression assay of OsNPF4.5 in response to AM symbiosis. FIG. 13A illustrates transcripts of OsNPF4.5 in different tissues of mycorrhizal (AM) and nonmycorrhizal (NM) plants. FIGS. 13B-D illustrate time-course expression of OsNPF4.5 and OsPT11 (used as a control) in rice mycorrhizal roots. FIG. 13D illustrates quantification of AM fungal colonization at different sampling time points. FIGS. 13E-F illustrate histochemical β-glucuronidase (GUS) staining of rice roots expressing pOsNPF4.5::GUS in the absence (FIG. 13E) and presence (FIG. 13F) of inoculation. FIG. 13G illustrates magenta-GUS staining of the mycorrhizal roots. FIG. 13H illustrates co-localization of GUS activity (indicated by the purple color, from the overlay of the Trypan Blue and Magenta-GUS stains). Red arrows indicate arbuscules; blue arrows denote non-colonized cells in mycorrhizal roots; and bars=50 μm.

FIGS. 14A-F illustrate functional characterization of OsNPF4.5 in vitro and in vivo. FIGS. 14A-B illustrate results of nitrate-uptake assay in Xenopus oocytes injected with OsNPF4.5 and CHL1 cRNAs using ¹⁵N-nitrate at a pH 5.5 (FIG. 14A) and a pH 7.4 (FIG. 14B);CHL1 was used as a positive control. FIG. 14C illustrates nitrate uptake kinetics of OsNPF4.5 in oocytes. OsNPF4.5 cRNA was injected into oocytes, which were incubated in the ND96 solution containing 0.25, 1, 2.5, 5, 10, 15, and 20 mM Na¹⁵NO₃, respectively, for 2 h at a pH 5.5. FIG. 14D illustrates current-voltage curves of oocytes expressing OsNPF4.5. The I-V curves shown were recorded from OsNPF4.5- and H₂O-injected oocytes, which were treated with 10 mM nitrate at a pH 5.5. Values are means±S.E. (n=10 oocytes). FIGS. 14E-F illustrate the ¹⁵N accumulation in roots of WT and OsNPF4.5-overexpressing plants under ¹⁵NO₃⁻ (FIG. 14E) or ¹⁵NH₄+(FIG. 14F) supply hydroponic conditions. In the uptake experiment, WT and OsNPF4.5-overexpressing transgenic lines, referred as OX lines, were suffered from N deprivation for 4 days, and then resupplied with ¹⁵N-labled 2.5 mM NO₃⁻ or 2.5 mM NH₄⁺ for 10 min. Values are means±S.E. of 5 biological replicates (n=5). The asterisks indicate significant differences, *P<0.05; **P<0.01, ***P<0.001.

FIGS. 15A-L illustrate physiological analysis of the OsNPF4.5 loss function mutants. WT and three osnpf4.5 mutant lines generated by CRISPR/Cas9 were cultivated in a compartmented growth system containing a middle root/hyphal compartment (RHC) that was separated by 30-mm nylon meshes from two hyphal compartments (HC). The RHC and HC were irrigated with 2.5 mM NO₃⁻ and ¹⁵NO₃⁻ weekly, respectively. The inoculated and mock-inoculated WT and osnpf4.5 plants were harvested for physiological analysis at 6 wpi. FIG. 15A illustrates shoot biomass (dry weight), shoot N content is illustrated in FIGS. 15B-C and ¹⁵N accumulation is illustrated in FIG. 15D of the WT and osnpf4.5 mutant plants inoculated with R. irregularis (AM) or mock-inoculated controls (NM). FIG. 15E illustrates the contribution of the symbiotic NO₃⁻ acquisition pathway to overall N uptake of WT and osnpf4.5 mutants. FIGS. 15F-L illustrate the mycorrhizal colonization level (FIG. 15F) determined in hypha (H), arbuscule (A), and vesicule (V), and arbuscule incidence and morphology in WT (FIG. 15G and FIG. 15K) and osnpf4.5 mutants (FIGS. 15H-J and FIG. 15L). Values are means±S.E. of 5 independent biological replicates (n=5). Different letters and asterisks indicate significant differences, *P<0.05; **P<0.01; bar, A-D, 50 μm; and E and F, 25 μm.

FIG. 16 illustrates a model for N uptake, assimilation and translocation in AM symbiosis. AM fungi can take up both NH₄⁺ and NO₃⁻, as well as organic N forms, such as amino acids (AAs) and small peptides (SPs), from soil solution via their extraradical mycelium (ERM). The NH₄⁺ in fungal cytoplasm can be rapidly assimilated into amino acids, mainly arginine, via the glutamine synthetase-glutamate synthase (GS-GOGAT) pathway, and translocated probably coupled with Poly-P through the intraradical hyphae. After hydrolysis in the arbuscule, NH₄⁺ is exported from the AM fungus to the periarbuscular space (PAS), and subsequently imported, probably in the form of NH₃, into the root cell, by the putative plant NH₄⁺ transporters located on the periarbuscular membrane (PAM). The NO₃⁻ absorbed by extraradical mycelium can be directly translocated into intraradical hyphae, and released into the interfacial apoplast. The import of NO₃⁻ into root cell is mediated by the PAM-localized NO₃⁻ transporters, such as OsNPF4.5. Legend: NR, nitrate reductase; NiR, nitrite reductase: GS, glutamine synthetase; GOGAT, glutamate synthase; AMT, ammonium transporter; and AAP, amino acid permease. Question marks and dotted lines indicate that the putative transporters or transport/metabolic processes have not yet been established.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Arbuscular mycorrhiza (AM) is a kind of beneficial fungi belonging to the genus Glomus spp., referred to as arbuscular mycorrhizal fungi (AM fungi). In the soil, AM fungi form a mutually beneficial symbiosis with the plant root system.

More than 85% of terrestrial plants on the earth, except cruciferous, liliaceous, caryophyllaceae, and a few legumes that form roots, can coexist with different types of AM fungi in the soil to form arbuscular mycorrhiza. After the formation of mycorrhiza, plants can absorb nutrients in the soil through two ways: (1) the direct absorption through the plant's own root system; and (2) the indirect absorption through the AM fungal mycelium, also known as the mycorrhizal pathway.

Plant roots can expand the absorption space in the soil dozens of times by means of the extra-root hyphae of AM fungi, increasing the absorption and utilization of nutrients, mainly P and N, in the soil. Phosphorus transporters and ammonia transporters that have been strongly/specifically expressed in mycorrhizal have been reported in a variety of species, such as, alfalfa, baimaigen, rice, and the like. However, nitrate transporters induced by mycorrhiza are rarely reported.

Rice is an important food crop in China. Although it has lived in a flooded environment for a long time, a large number of studies have proven that nitrate plays a vital role in the growth and development of rice. AM fungi are aerobic fungi, and long-term flooding will directly affect the formation of mycorrhizal symbionts. However, the oxygen secretion function of rice aeration tissues causes rice to still be infested by indigenous AM fungi.

Studies have shown that flooding irrigation for seven consecutive days on dry farming rice can reduce the infection rate of AM fungi on the root system, but it cannot completely prevent mycorrhizal symbiosis. Studies have found that a large number of different types of AM fungal spores also exist in paddy soil. In addition, during the rice growth period, moderate sun exposure is required to improve the soil environment, and enhance root vitality and microbial activity, to achieve the purpose of improving nutrient absorption and reducing ineffective tillers. This also provides AM fungi for infecting rice roots and NO₃⁻ favorable environments.

In sum, a need exists for more effective absorption and utilization of nitrogen nutrients in plants. Various embodiments of the present disclosure address the aforementioned need.

Nitrate Transporter Genes

In some embodiments, the present disclosure pertains to nitrate transporter gene OsNPF4.5. In some embodiments, the nitrate transporter gene includes the nucleotide sequence shown in SEQ ID NO:1 or a functional variant thereof.

In some embodiments, the nitrate transporter gene includes the nucleotide sequence shown in SEQ ID NO:1. In some embodiments, the nitrate transporter gene includes the functional variant of the nucleotide sequence shown in SEQ ID NO:1. In some embodiments, the functional variant has at least 65% sequence identity to SEQ ID NO:1. In some embodiments, the functional variant has at least 75% sequence identity to SEQ ID NO:1. In some embodiments, the functional variant has at least 80% sequence identity to SEQ ID NO:1. In some embodiments, the functional variant has at least 85% sequence identity to SEQ ID NO:1. In some embodiments, the functional variant has at least 90% sequence identity to SEQ ID NO:1. In some embodiments, the functional variant has at least 95% sequence identity to SEQ ID NO:1. In some embodiments, the functional variant has at least 99% sequence identity to SEQ ID NO:1.

In some embodiments, functional variants of SEQ ID NO:1 are orthologs of SEQ ID NO:1. In particular, SEQ ID NO:1 represents the rice nitrate transporter gene OsNPF4.5. As such, in some embodiments, functional variants of SEQ ID NO:1 represent nitrate transporter gene OsNPF4.5 in different species. In some embodiments, the different species include, without limitation, Medicago (MtNPF4.5), maize (ZmNPF4.5) and sorghum (SbNPF4.5).

The nitrate transporter genes of the present disclosure can be in various forms. For instance, in some embodiments, the nitrate transporter genes of the present disclosure include optimized codons suitable for expression in one or more plants. In some embodiments, the nitrate transporter genes of the present disclosure are in isolated form. In some embodiments, the nitrate transporter genes of the present disclosure are in cDNA form. In some embodiments, the isolated nitrate transporter genes of the present disclosure are isolated from their native environments. In some embodiments, the native environments include, without limitation, cells, chromosomes, or combinations thereof.

In some embodiments, the nitrate transporter genes of the present disclosure are capable of being introduced into a plant for expression of the nitrate transporter protein OsNPF4.5. In some embodiments, the nitrate transporter genes of the present disclosure are contained in a plant. In some embodiments, the nitrate transporter genes of the present disclosure are contained in a plant as an exogenous gene. In some embodiments, the nitrate transporter genes of the present disclosure are contained in a plant as an overexpressed gene.

Recombinant Expression Vectors

In some embodiments, the nitrate transporter genes of the present disclosure are contained in a recombinant expression vector. Accordingly, additional embodiments of the present disclosure pertain to recombinant expression vectors that include the nitrate transporter genes of the present disclosure.

The nitrate transporter genes of the present disclosure may be contained in various recombinant expression vectors. For instance, in some embodiments, the recombinant expression vector includes, without limitation, a plasmid, an Ri plasmid, a Ti plasmid, a plant virus vector, or combinations thereof. In some embodiments, the recombinant expression vector is a plasmid.

In some embodiments, the recombinant expression vector includes a promoter that is operable for facilitating the transcription of the nitrate transporter gene in one or more plants. In some embodiments, the promoter includes, without limitation, cauliflower mosaic virus (CAMV) 35S promoter, Ubiquitin promoter, or combinations thereof.

In some embodiments, the recombinant expression vectors of the present disclosure can include enhancers, such as transcription enhancers or translation enhancers. In some embodiments, the recombinant expression vectors of the present disclosure can also include genes for enzymes that can be used to confer antibiotic resistance, color change (e.g., (β-glucuronidase), or luminescence (e.g., luciferase).

Nitrate Transporter Proteins

Additional embodiments of the present disclosure pertain to nitrate transporter protein OsNPF4.5. In some embodiments, the nitrate transporter protein includes the amino acid sequence shown in SEQ ID NO: 2 or a functional variant thereof.

In some embodiments, the nitrate transporter proteins of the present disclosure include the amino acid sequence shown in SEQ ID NO:2. In some embodiments, the nitrate transporter proteins of the present disclosure include the functional variant of the amino acid sequence shown in SEQ ID NO:2. In some embodiments, the functional variant has at least 65% sequence identity to SEQ ID NO:2. In some embodiments, the functional variant has at least 75% sequence identity to SEQ ID NO:2. In some embodiments, the functional variant has at least 80% sequence identity to SEQ ID NO:2. In some embodiments, the functional variant has at least 85% sequence identity to SEQ ID NO:2. In some embodiments, the functional variant has at least 90% sequence identity to SEQ ID NO:2. In some embodiments, the functional variant has at least 95% sequence identity to SEQ ID NO:2. In some embodiments, the functional variant has at least 99% sequence identity to SEQ ID NO:2.

In some embodiments, functional variants of SEQ ID NO:2 are orthologs of SEQ ID NO:2. In particular, SEQ ID NO:2 represents the rice nitrate transporter protein OsNPF4.5. As such, in some embodiments, functional variants of SEQ ID NO:2 represent nitrate transporter protein OsNPF4.5 in different species. In some embodiments, the different species include, without limitation, Medicago, maize, and sorghum.

The nitrate transporter proteins of the present disclosure may be in various forms. For instance, in some embodiments, the nitrate transporter proteins of the present disclosure may be in isolated form. In some embodiments, the nitrate transporter proteins of the present disclosure may be in purified form. In some embodiments, the nitrate transporter proteins of the present disclosure may be contained in a plant as an exogenous protein.

Methods of Enhancing Plant Growth

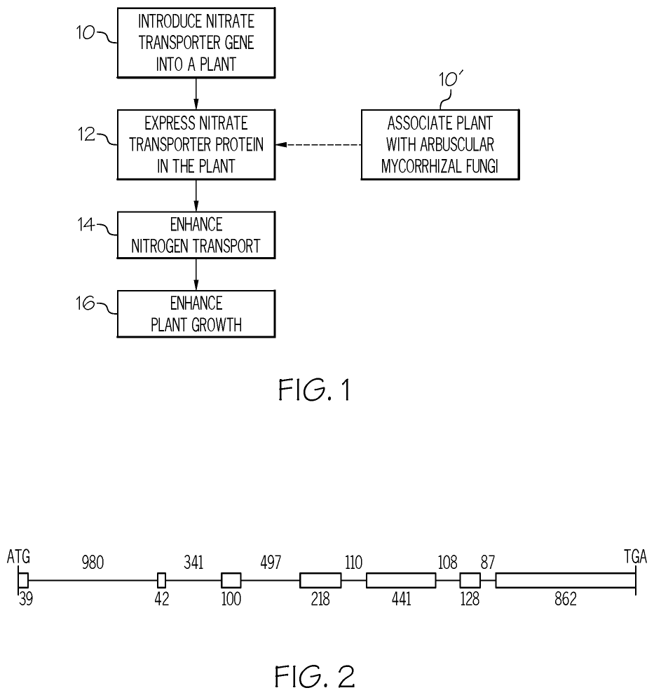

Additional embodiments of the present disclosure pertain to methods of enhancing a plant's growth. For instance, in some embodiments illustrated in FIG. 1, the methods of the present disclosure include introducing one or more nitrate transporter genes of the present disclosure into the plant (step 10) to result in the expression of one or more nitrate transporter proteins of the present disclosure in the plant (step 12). In some embodiments, the expression of the one or more nitrate transporter proteins enhances nitrogen transport (step 14), which in turn enhances plant growth (step 16). In some embodiments, the methods of the present disclosure also include a step of associating the plant with an arbuscular mycorrhizal fungi (step 10′).

As set forth in more detail herein, the methods of the present disclosure can have numerous embodiments. For instance, various methods may be utilized to introduce various nitrate transporter genes into various plants in order to enhance plant growth in various manners. Various methods may also be utilized to associate arbuscular mycorrhizal fungi with plants.

Introduction of Nitrate Transporter Genes into Plants

Various methods may be utilized to introduce nitrate transporter genes into plants. For instance, in some embodiments, introduction occurs by methods that include, without limitation, gene gun introduction methods, agrobacterium-mediated introduction methods, pollen tube channel introduction methods, or combinations thereof.

Enhanced Plant Growth

Without being bound by theory, the methods of the present disclosure may enhance plant growth through various mechanisms. For instance, in some embodiments, the introduced and expressed nitrate transporter protein OsNPF4.5 enhances plant growth by enhancing the plant's absorption of nitrogen. In some embodiments, the expressed nitrate transporter protein OsNPF4.5 enhances the plant's absorption of nitrogen by enhancing the transport of nitrate into the plant.

The methods of the present disclosure may enhance plant growth at various levels. For instance, in some embodiments, the methods of the present disclosure enhance plant growth by at least 25% relative to plants without the introduced nitrate transporter gene OsNPF4.5. In some embodiments, the methods of the present disclosure enhance plant growth by at least 50% relative to plants without the introduced nitrate transporter gene OsNPF4.5. In some embodiments, the methods of the present disclosure enhance plant growth by at least 65% relative to plants without the introduced nitrate transporter gene OsNPF4.5. In some embodiments, the methods of the present disclosure enhance plant growth by at least 100% relative to plants without the introduced nitrate transporter gene OsNPF4.5.

Enhanced plant growth may be represented in various manners. For instance, in some embodiments, enhanced plant growth is represented by an increase in height. In some embodiments, enhanced plant growth is represented by an increase in width. In some embodiments, enhanced plant growth is represented by an increase in the size of leaves. In some embodiments, enhanced plant growth is represented by an increase in total weight.

Association of Arbuscular Mycorrhizal Fungi with Plants

In some embodiments, the methods of the present disclosure also include a step of associating arbuscular mycorrhizal fungi with plants. In some embodiments, the association enhances the expression of endogenous nitrate transporter proteins in the plant, which in turn further enhances nitrogen transport and plant growth.

Various methods may also be utilized to associate arbuscular mycorrhizal fungi with plants. For instance, in some embodiments, the associating occurs by inoculating roots of the plant with the arbuscular mycorrhizal fungi.

Plants

The nitrate transporter genes and proteins of the present disclosure may be contained in various plants. Additionally, the methods of the present disclosure may be utilized to introduce the nitrate transporter genes of the present disclosure into various plants. Additional embodiments of the present disclosure include genetically modified plants that include an introduced nitrate transporter gene of the present disclosure.

In some embodiments, the plants of the present disclosure include, without limitation, monocotyledonous plants, dicotyledonous plants, or combinations thereof. In some embodiments, the plants of the present disclosure include, without limitation, rice, corn, soybean, cotton, tobacco, wheat, Medicago, maize, sorghum, and combinations thereof. In some embodiments, the plants of the present disclosure include rice.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Rice Nitrate Transporter Gene Specifically Induced by Arbuscular Mycorrhiza

This Example describes a nitrate transporter gene specifically induced by rice arbuscular mycorrhiza and its application. The first nitrate transporter gene OsNPF4.5, specifically induced by arbuscular mycorrhizal in monocotyledonous plants (which was identified from rice), and the relationship between it and mycorrhizal symbiosis was studied. The use of OsNPF4.5 genes were proposed for a method of improving the absorption and utilization of nitrogen nutrients in the symbiotic process of rice (upland rice) and beneficial microorganisms arbuscular mycorrhizal fungi.

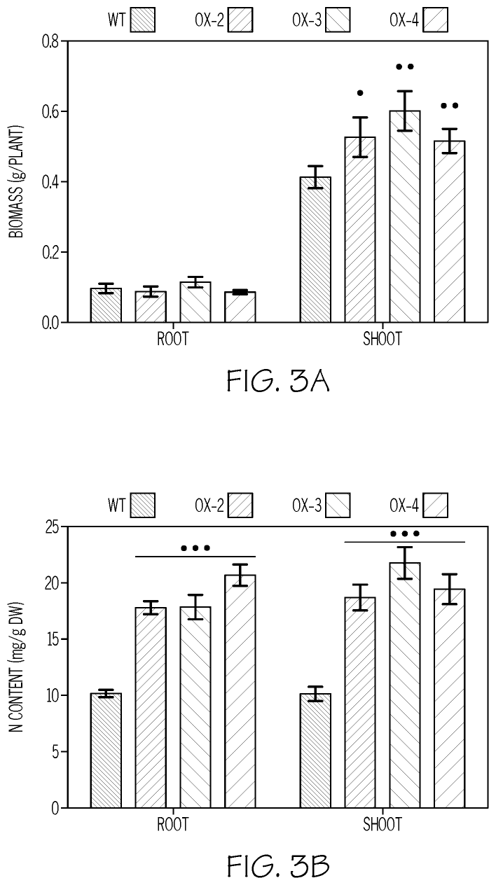

This Example discloses a sequence structure (FIG. 2) of nitrate transporter gene (OsNPF4.5 gene) specifically induced by rice mycorrhiza and its encoded protein. This gene comes from rice (Oryza sativa L.) and can be introduced into plants as a target gene to increase the plant's absorption of nitrogen for plant variety improvement (FIGS. 3A and 3B). The encoded protein has the function of transporting nitrate.

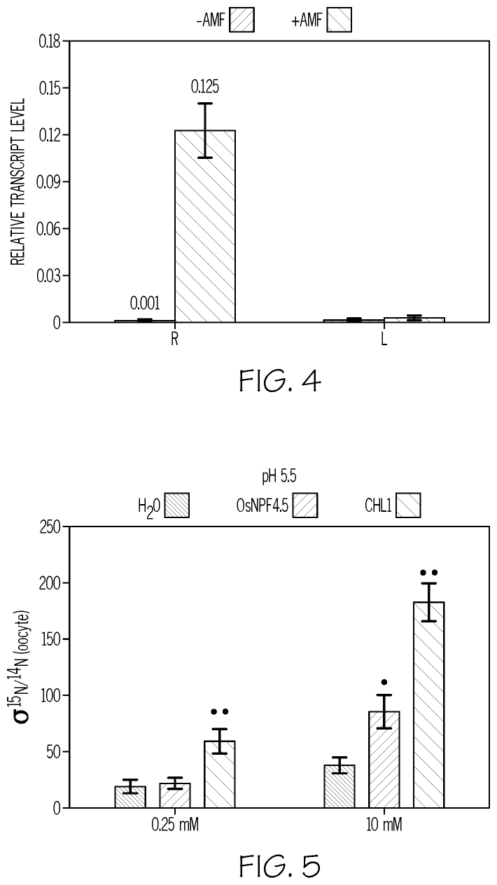

The function of the OsNPF4.5 gene is to participate in the process of infection and symbiosis between plants and beneficial microorganisms arbuscular mycorrhizal fungi. Transcription level analysis indicates that the OsNPF4.5 gene is specifically induced and expressed by arbuscular mycorrhizal (FIG. 4).

The OsNPF4.5 gene in this Example was derived from rice and has optimized codons suitable for expression of monocotyledonous plants, such as, but not limited to, rice. Its genetically engineered recipient plants are more suitable for rice and corn than dicotyledonous plants, such as, soybean, cotton, tobacco, wheat and other monocotyledons.

The OsNPF4.5 gene in this Example is used as a target gene to construct a plant expression vector, where any promoter, such as, cauliflower mosaic virus (CAMV) 35S promoter, ubiquitin promoter or self-promoter can be used, and the expression vector can include enhancers, whether they are transcription enhancers or translation enhancers. To simplify the identification of transformed cells, enzymes can be used that include selectable markers, including antibiotic resistance, or compounds that can be identified by color change (e.g., B-glucuronidase; GUS) or luminescence (e.g., luciferase). Classes can also be selected without marking. The expression vector Ti plasmid, Ri plasmid, plant virus vectors, and the like can be used. The transformation method can use an agrobacterium-mediated method, a gene gun method, a pollen tube channel method, or other methods to transform plants.

### Example 1.1. Molecular Cloning of OsNPF4.5— A Nitrate Transporter Gene Induced by Rice Mycorrhiza

The rice variety “Nipponbare” (conventional experimental variety) was selected for this Example. Before testing, sand was sterilized by dry heat at 180 degrees and placed in a 4 liter pot. Four pots per pot (2 plants per pot) were planted with rice seedlings that grew for two weeks after germination, and about 200 spores of R. irregularis fungus or inactivated fungus (as a control) were inserted around the root of each pot. After pouring a rice International Rice Research Institute (IRRI) nutrient solution (phosphorus concentration reduced to 30 μM) once a week for six weeks, root samples were taken and frozen in liquid nitrogen. The root system was taken apart, ground with a mortar, and added to a 1.5 mL Eppendorf (EP) tube containing lysate, shaken thoroughly, and then moved into a glass homogenizer. After homogenization, the sample was moved to a 1.5 mL EP tube and total RNA (TRIzol Reagents, Invitrogen, USA) was extracted. Formaldehyde denaturing gel electrophoresis was used to identify the total RNA quality, and then the RNA content was determined on a spectrophotometer.

Because OsNPF4.5 has very low expression in rice roots not inoculated with AM fungi, and its full-length coding sequence cannot be found in an expressed sequence tags/complementary DNA (EST/cDNA) library, Applicants used rapid amplification of cDNA ends-polymerase chain reaction (RACE-PCR) using Ambion's RACE kit (FirstChoice RLM-RACE Kit, Ambion, Inc., Austin, Tex., USA) technology, and cloned the full-length cDNA sequence of this gene from rice mycorrhiza. First, using the RNA in the above as a template and Oligo (dT) as a locking primer, the first strand of cDNA was synthesized by reverse transcription under the action of the reverse transcriptase MMLV. Then, the universal primer UMP containing part of the linker was used as the upstream primer and the gene-specific primer GSP1 was used as the downstream primer. The first strand of cDNA was used as a template for PCR cycles to amplify the cDNA fragment at the 5′ end of the target gene. Similarly, UMP was used as the downstream primer and GSP2 was used as the upstream primer to amplify the 3′ end cDNA fragment. Finally, full-length cDNA was obtained from two 3′/5′-RACE products with overlapping sequences. The cDNA sequence of the rice nitrate transporter gene OsNPF4.5 was obtained by sequencing. Sequence analysis showed that the open reading frame (ORF) of this gene is 1830 bp, and there are 6 introns in the coding region.

Because OsNPF4.5 belongs to the NRT1/PTR family, it indicates that it may have a nitrate transport function. In order to confirm its function, the cDNA sequence was cloned and connected to a frog egg expression vector pT7 Ts to synthesize cRNA in vitro. The cRNA of OsNPF4.5 was injected into the frog egg body after 48 hours of incubation and placed in a medium containing 0.25 mM and 2.5 mM, respectively, treated with ¹⁵NO₃⁻ for 2 hours to detect ¹⁵N abundance in the frog eggs. The analysis result of the frog egg experiment proves that the gene newly obtained from rice is indeed a gene encoding nitrate transporter (FIG. 5). The rice nitrate transporter gene OsNPF4.5 of this Example is the first reported in rice, and it is also the first nitrate transporter gene related to arbuscular mycorrhizal symbiosis found in plants, which is expected to be applied to plants, especially dry farming plants, to improve the absorption and utilization of nitrogen nutrients during the arbuscular mycorrhizal symbiosis.

### Example 1.2. Sequence Information and Characteristic Analysis of OsNPF4.5

The OsNPF4.50RF (open reading frame) of this Example is 1830 bp (SEQ ID NO. 3) and contains 7 exons and 6 introns. DNAssist software analysis shows that OsNPF4.5 encodes a total of 609 amino acids and has 12 transmembrane domains, which is consistent with the basic characteristics of transport proteins. The comparison of the Blast program revealed that the nucleotide sequence of the OsNPF4.5 gene was 78.2% and 78.1% with the sorghum SbNPF4.3 and maize ZmNPF4.5 nucleotide similarities, respectively. This indicates that the OsNPF4.5 gene is highly conserved among different species in evolution.

### Example 1.3. Study on the Expression of OsNPF4.5

The primers at both ends of OsNPF4.5 were designed using the sequence in Example 1.1 for quantitative reverse transcription (RT)-PCR to analyze the expression of shoots and shoots of rice seedlings inoculated with arbuscular mycorrhizal fungi of the rice actin gene Rac 1, the expression of which was used as an internal reference. The results show that OsNPF4.5 is very low in the aboveground and uninoculated roots, but it is strongly induced specifically in the roots inoculated with arbuscular mycorrhizal fungi by hundreds of times when compared to the control.

The primers used for quantitative RT-PCR are as follows:

OsACTIN QF:
(SEQ ID NO: 4)
CAACACCCCTGCTATGTACG
OsACTIN QR:
(SEQ ID NO: 5)
CATCACCAGAGTCCAACACAA
OsNPF4.5 QF:
(SEQ ID NO: 6)
CGCCGTGCTCAGCTTCCTCAACTT
OsNPF4.5 QR:
(SEQ ID NO: 7)
AGGCAAAAATGGTAGCAACAACTG

### Example 1.4. Obtaining OsNPF4.5 Transgenic Rice Plants

According to the full-length sequence of OsNPF4.5 obtained in Example 1.1, a primer designed to amplify the complete reading frame was designed and restriction enzyme sites were introduced on the upstream and downstream primers, respectively (this may depend on the vector selected), in order to construct an expression vector.

OsNPF4.5 OF:
(SEQ ID NO: 8)
cgcGTCGACATGAGCAAAGTAACTCAAGCTA
(underlined indicates the cleavage site)
OsNPF4.5 OR:
(SEQ ID NO: 9)
gccAAGCTTTCATACTTTGTGCTCTGCTG
(underlined indicates the cleavage site)

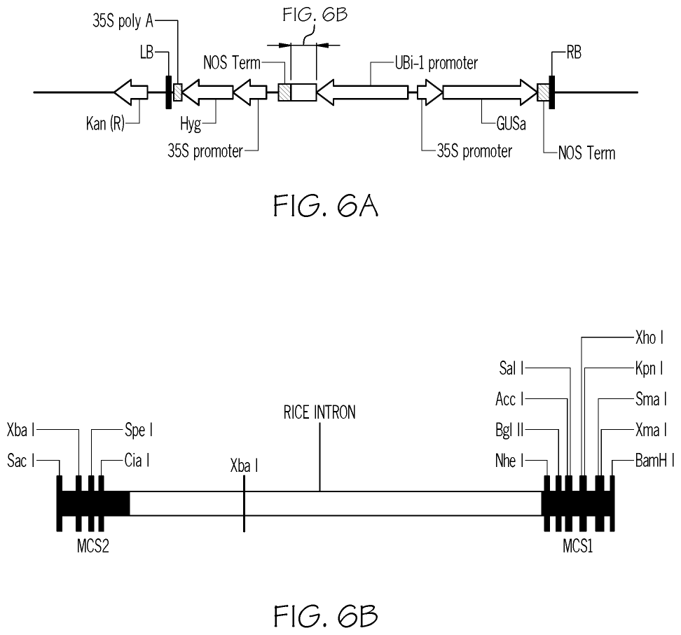

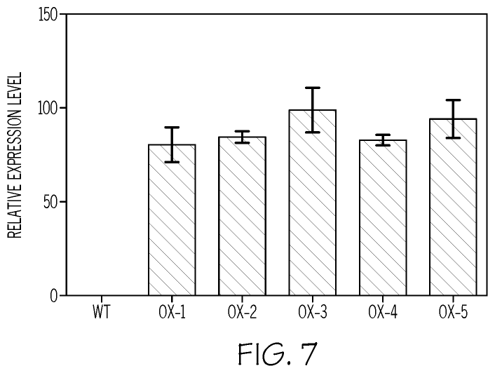

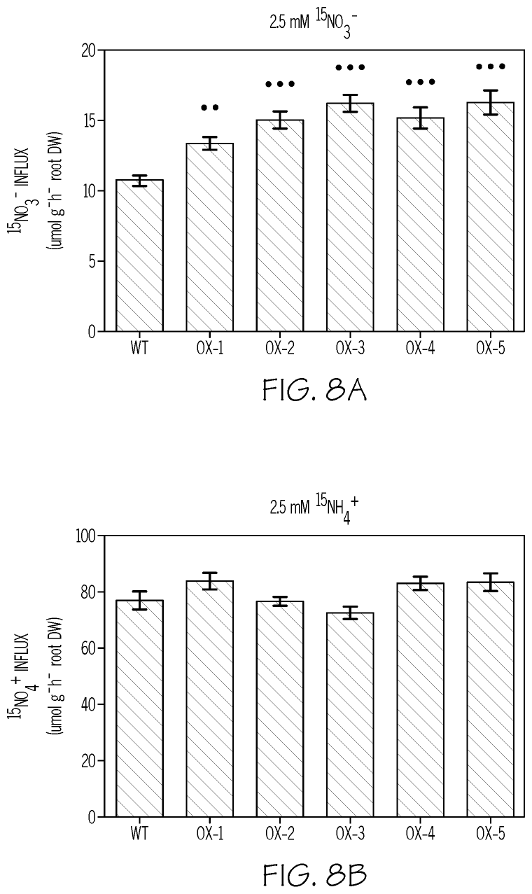

Using the amplification product obtained in Example 1.1 as a template, after PCR amplification, the cDNA of OsNPF4.5 was cloned into the intermediate vector pGEM-T, and further cloned into the commonly used binary expression vector pTCK303 (FIGS. 6A and 6B). A good expression vector was identified under the premise of a correct reading frame, then transferred into agrobacterium, and then transferred to the rice variety Nipponbare. The transgenic plants to be obtained were subjected to functional verification after verifying the overexpression effect by quantitative RT-PCR (FIG. 7). The T2 generation of the transgenic plants and the control plants were treated with 2.5 mM ¹⁵NO₃⁻ and ¹⁵NH₄⁺, and their ¹⁵N absorption rate was detected. The results showed that the ¹⁵N absorption rate of transgenic rice under ¹⁵NO₃⁻ treatment was significantly higher than that of the control group, but there was no difference under ammonia treatment (FIGS. 8A and 8B).

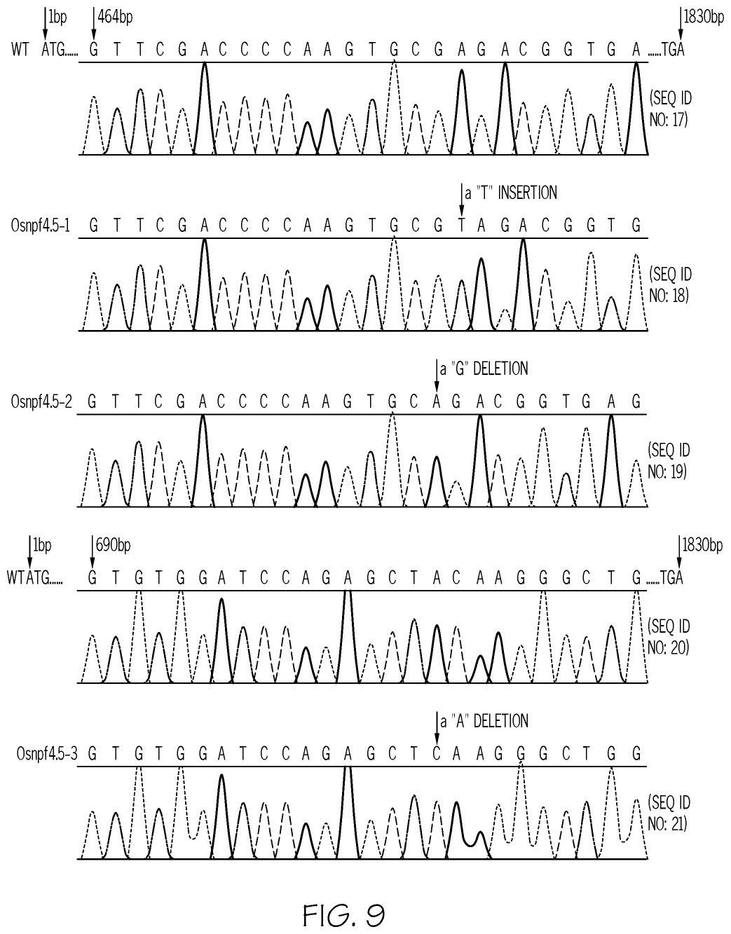

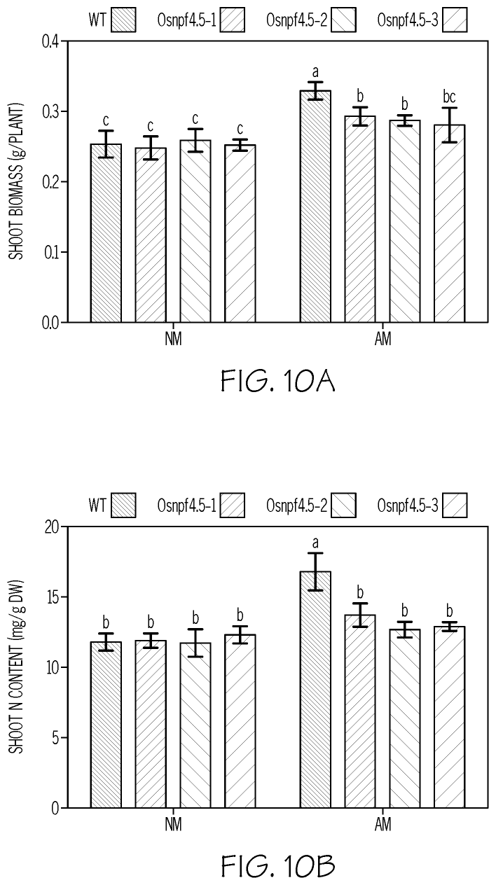

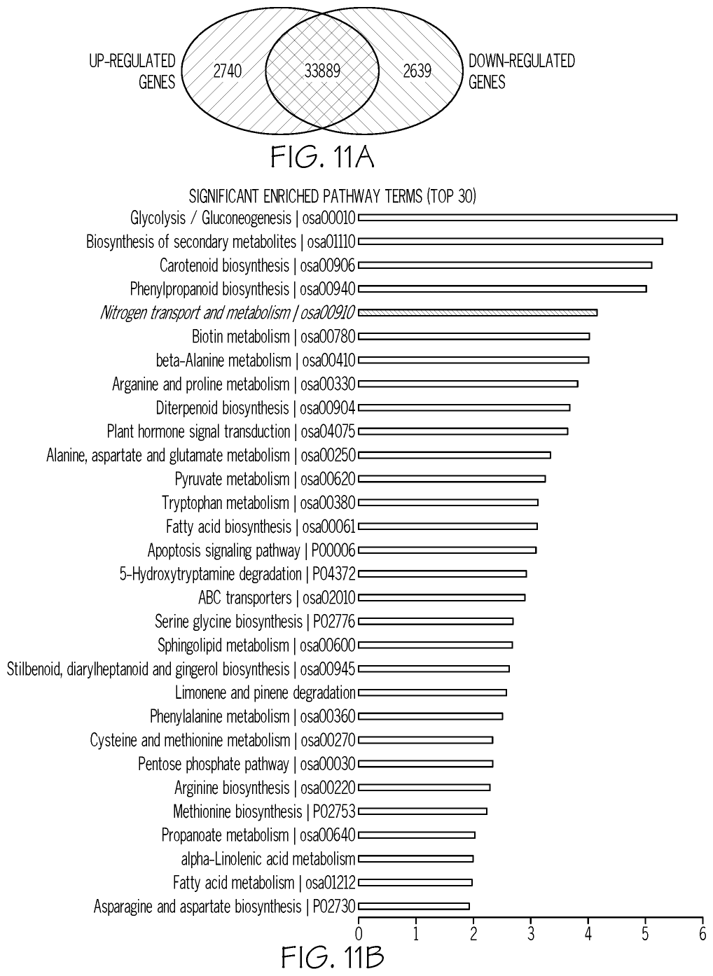

In order to better study its function in plants, Applicants used CRISPR-Cas9 technology to create Osnpf4.5 mutant materials. First, three specific spacers were designed in the coding region of OsNPF4.5 and connected to sgRNA and Cas9 vectors. Then they were transferred to agrobacterium and the transferred to the rice variety Nipponbare. The homozygosity of the three strains were obtained through sequencing and identification. The three mutants were osnpf4.5-1, osnpf4.5-2, and osnpf4.5-3 (FIG. 9). In the case of arbuscular mycorrhizal fungi, the osnpf4.5 mutant transgenic plants had significantly lower aerial biomass and nitrogen concentration compared with the wild type (FIGS. 10A and 10B).

### Example 1.5. Conclusion

The above Examples show that the nitrate transporter gene OsNPF4.5 specifically induced and expressed by the cloned rice mycorrhiza is the mycorrhizal induced nitrate transporter gene, cloned for the first time in rice. The above Examples shows that this gene is closely related to mycorrhizal symbiotic nitrate absorption. Because this gene is significantly induced by arbuscular mycorrhizal fungi, it is more suitable for genetic improvement of stress resistance (nutrient stress) of many food crops, such as, but not limited to, upland rice, corn, wheat, and so on.

In this Example, Applicants cloned a cDNA from the monocotyledonous rice (Oryza sativa L.), which encodes a nitrate transporter, and is named OsNPF4.5. mRNA expression analysis showed that OsNPF4.5 specifically induced expression only in the root system inoculated with mycorrhiza, but the expression level was very low in the root system and aerial part without inoculation. The transgenic study in this Example showed that the OsNPF4.5 gene was transferred into rice, and the ¹⁵N₃⁻ absorption rate of transgenic rice was significantly higher than that of the control group under ¹⁵NO₃⁻ treatment, but there was no difference under ammonia treatment. Osnpf4.5 mutant transgenic plants were inoculated with arbuscular mycorrhizal fungi, compared with the wild type, and the aboveground biomass and nitrogen concentration of the mutant were significantly reduced, while the root infection rate and arbus abundance were also reduced accordingly.

In methods of this Example, the OsNPF4.5 gene can be used as the target gene to construct a plant expression vector, where any promoter such as cauliflower mosaic virus (CAMV) 35S promoter, ubiquitin promoter or mycorrhiza-specific induced promoter can be used if necessary, and the expression vector may include an enhancer, whether it is a transcription enhancer or a translation enhancer. To simplify the identification of transformed cells, enzymes can be used that include selectable markers including antibiotic resistance, or compounds that can be identified by color change (e.g., B-glucuronidase; GUS) or luminescence (e.g., luciferase). Classes can also be selected without marking. As the expression vector, Ti plasmid, Ri plasmid, plant virus vector, and the like can be used. The transformation method can use an agrobacterium-mediated method, a gene gun method, a pollen tube channel method, or other methods to transform plants.

### Example 2. Functional Analysis of the OsNPF4.5 Nitrate Transporter Reveals a Conserved Mycorrhizal Pathway of Nitrogen Acquisition in Plants

This Example describes functional analysis of the OsNPF4.5 nitrate transporter gene. The findings reveal a conserved mycorrhizal pathway of nitrogen acquisition in plants.

Low availability of nitrogen (N) is often a major limiting factor to crop yields in most nutrient-poor soils. Arbuscular mycorrhizal (AM) fungi are beneficial symbionts of most land plants that enhance plant nutrient uptake, in particularly of phosphate. A growing number of reports point to the substantially increased N accumulation in many mycorrhizal plants. However, the contribution of AM symbiosis to plant N nutrition and the mechanisms underlying the AM-mediated N acquisition are still in the early stages of being understood.

Here, Applicants report that inoculation with AM fungus Rhizophagus irregularis remarkably promoted rice (Oryza sativa) growth and N acquisition, and about 42% of the overall N acquired by rice roots could be delivered via the symbiotic route under N—NO₃⁻ supply condition. Mycorrhizal colonization strongly induced expression of the putative nitrate transporter gene OsNPF4.5 in rice roots, and its orthologues ZmNPF4.5 in Zea mays and SbNPF4.5 in Sorghum bicolor. OsNPF4.5 is expressed in the cells containing arbuscules and displayed a low-affinity NO₃⁻ transport activity when expressed in Xenopus laevis oocytes. Moreover, knock out of OsNPF4.5 resulted in a 45% decrease in symbiotic N uptake and a significant reduction in arbuscule incidence when supplied NO₃⁻ as a N source. Based on Applicants' results, Applicants propose that NPF4.5 plays a role in mycorrhizal NO₃⁻ acquisition, a symbiotic N uptake route that might be highly conserved in gramineous species.

Low availability of nitrogen (N), mainly nitrate in aerobic soils, is a primary limiting factor for crop production. Most terrestrial plants live in symbiosis with arbuscular mycorrhizal (AM) fungi to increase nutrient uptake, including N, from soil. Research in the AM symbiosis field has focused almost exclusively on ammonium as the form of N transferred to the plants, and there has been no direct evidence of N transfer as nitrate thus far. Here, Applicants also report that mycorrhizal rice could receive more than 40% of its N via the mycorrhizal pathway and that the AM-specific nitrate transporter OsNPF4.5 accounted for approximately 45% of the mycorrhizal nitrate uptake. This Example suggests the presence of a mycorrhizal route for nitrate uptake in plants.

### Example 2.1. Introduction

In natural soil ecosystem, the majority of land plants can form mutualistic symbiosis with arbuscular mycorrhizal (AM) fungi of Glomeromycotina to better adapt to limited nutrient supplies. AM association is an endosymbiotic process that requires the differentiation of both symbionts to create novel contact interfaces within the cells of plant roots. In the AM symbiosis, the fungal hyphae penetrate the root epidermis, grow through the intercellular spaces of the root and subsequently invade cortical cells, developing highly branched, tree-like structures called arbuscules.

Cortical cells develop a specialized membrane, the periarbuscular membrane (PAM), to envelop each branching hypha to separate the fungus from the plant cell cytoplasm, resulting in an extensive plant-fungal interface specialized for nutrient exchange. Upon the formation of AM symbiosis, mycorrhizal plants have two pathways for nutrient uptake, either direct uptake from the soil via root hairs and root epidermis, or indirectly through the AM fungal hyphae at the plant-fungus interface. It has been demonstrated that AM fungi dominates Pi uptake in symbiotic plants.

Nitrogen (N) is an important nutrient for plant growth and development. The primary forms of N absorbed by plant roots are nitrate (NO₃⁻) in aerobic upland soil and ammonium (NH₄±) in flooding soil. An increasing number of reports suggest that AM fungi can take up both NO₃⁻ and NH₄⁺, as well as organic N forms from the surrounding soils. Although N transfer in the AM symbiosis has been receiving increasing attention, the mechanism underlying the AM-mediated N acquisition pathway remains largely unknown. Current data proposes that once N has been transported into the fungal cytoplasm, it is assimilated into arginine, translocated probably together with Poly-P through the intraradical hyphae, and after hydrolysis in the arbuscule, NH₄⁺ is exported from the AM fungus to the periarbuscular space.

The import of NH₄⁺ across the PAM, probably in the form of NH₃, into the root cell is then mediated by plant NH₄⁺ transporters (AMTs). In some mycorrhizal plants living in aerobic environments examined so far, such as Medicago truncatula, Lotus japonicus, Glycine max and Sorghum bicolor, two to five AMT transporters were found to be specifically expressed or strongly upregulated in mycorrhizal roots. Immunolocalization evidence showed that two mycorrhiza-induced AMTs, GmAMT4.1 and SbAMT3.1, from G. max and S. bicolor, respectively, localize exclusively on the PAM, strongly suggesting the existence of a symbiotic NH₄⁺ uptake pathway at least in these plant species.

Nonetheless, AM association occurs preferably in aerobic soil condition, in which NO₃⁻ is the major form of inorganic N, due to rapidly nitrification of NH₄±. Therefore, it is possible that a symbiotic pathway for NO₃⁻ uptake that could be more important and/or prevalent than the mycorrhizal NH₄⁺ uptake route exists at least in some plant species.

Consistent with this notion, previous studies through transcriptome hunting have showed the presence of putative NO₃⁻ transporter genes with AM-induced expression in several plant species. However, it is still unclear whether NO₃⁻ could be directly translocated from the extraradical hyphae to the fungal structures within roots and whether NO₃⁻ could be directly transferred across the intraradical symbiotic interface into the root cells. This lack of knowledge restricts understanding regarding both the global N underground movement and the nutrient exchange capacity of what is arguably the world's most ancient, widespread, and important symbiosis.

Rice (Oryza sativa), a semi-aquatic crop plant that can grow in both flooding paddy and upland conditions, is one of the most important food crops worldwide. As most vascular flowering plants, rice has also inherited the capacity to be well colonized by AM fungi under aerobic growth conditions. Moreover, evidence from different research groups showed enhanced biomass production of rice plants inoculated with AM fungi. Because of the availability of technology to produce gene knockouts and overexpressing lines of specific genes, rice is a good model system to study the role of mycorrhizal N uptake routes on plant growth and the symbiotic interaction.

Here, Applicants report that about 42% of the overall N acquired by rice roots could be delivered via the symbiotic route under N—NO₃⁻ supply conditions, in which the mycorrhizal root-specific OsNPF4.5 nitrate transporter plays a crucial role. Applicants also report that by repressing NO₃⁻ transport across the intraradical symbiotic interface in loss of function osnpf4.5 mutants, decreases AM colonization efficiency and reduces arbuscule incidence.

### Example 2.2. RNA Sequencing Uncovered the Upregulation of Multiple Genes Involved in Nitrate Transport and Metabolism in Mycorrhizal Rice Plants

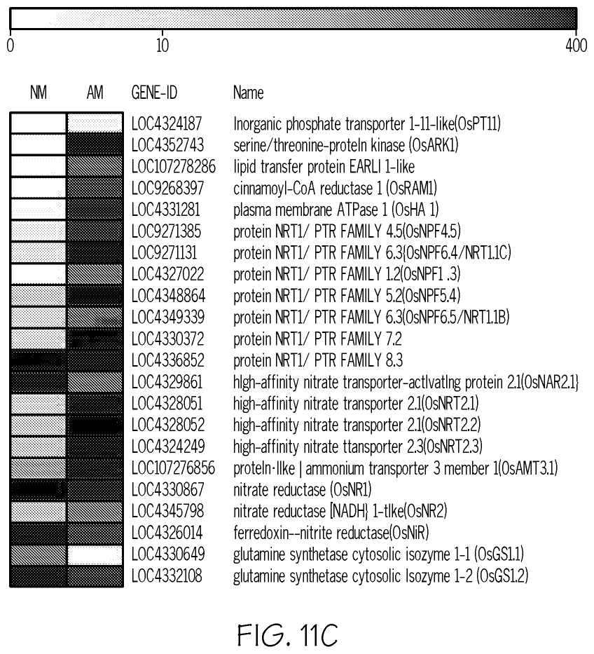

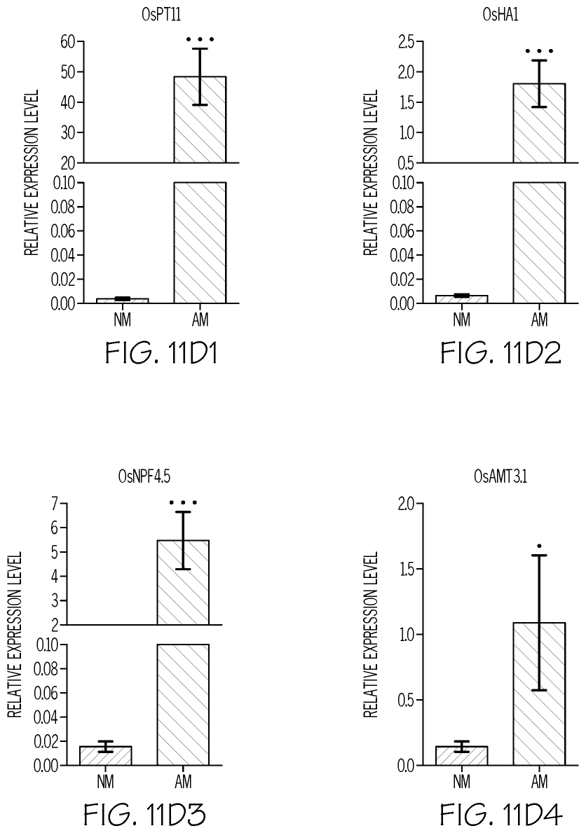

To gain an overview of rice transcriptional responses to AM fungal colonization, an ILLUMINA™ HiSeq2500 sequencing platform was used to conduct high-throughput RNA-seq analysis of both mycorrhizal and non-mycorrhizal roots collected from wild-type rice plants (O. sativa cv. Nipponbare) inoculated or mock-inoculated with Rhizophagus irregularis for 6 weeks. Differentially expressed genes (DEGs) between the two treatments were identified applying a P-value<0.05 and a two-fold change threshold. RNA-seq analysis revealed a total of 5379 DEGs, of which 2740 genes were upregulated and 2639 genes were downregulated in the rice mycorrhizal roots, whereas 33889 genes did not show significant alteration in transcript levels (FIG. 11A).

To better understand the potential functions of these DEGs and their related biological processes, Kyoto Encyclopedia of Genes and Genomes (KEGG) enrichment analysis were performed for upregulated genes (FIG. 11B). The Glycolysis/Gluconeogenesis pathway was found to be the most significantly enriched pathway, followed by pathways for biosynthesis of secondary metabolites, carotenoid biosynthesis, and phenylpropanoid biosynthesis. Interestingly, the N metabolism pathway was identified as the fifth most predominant enriched pathway in the KEGG analysis (FIG. 11B), with a ranking higher than the pathway of fatty acid biosynthesis. Several components involved in fatty acid biosynthesis and transport have been shown to be highly upregulated in the AM fungal-colonized roots, and play an essential role in maintaining AM symbiosis, through modulating lipid export from the host plant to AM fungi.

Careful scrutiny of the DEGs uncovered the substantial upregulation (2 to 500 folds) of 14 genes involved in NO₃⁻ transport and metabolism in rice mycorrhizal roots. Ten of these genes encode putative nitrate transporters from the NRT1/NPF and NRT2 families, of which OsNPF4.5 was the strongest up-regulated gene from a barely detectable expression level in nonmycorrhizal roots. Applicants found that OsNPF4.5 as substantially AM-induced genes could also be traced in a previously released microarray data of rice mycorrhizal roots, in which only 256 genes showing more than a three-fold change were detected. The AM-upregulated expression nature of some rice NPF genes, including the OsNPF4.5, was confirmed in a recent study.

Among the other four genes related to NO₃⁻ transport or metabolism, one encodes the high-affinity nitrate transporter-activating protein, OsNAR2.1, and the remaining three encode two putative nitrate reductases (NR) and a nitrite reductase (NiR), respectively (FIG. 11C). Comparing Applicants' data with the previously released microarray data of rice mycorrhizal roots, Applicants found only one common DEG encoding a putative ammonium transporter OsAMT3.1, with an 11-fold upregulation in rice mycorrhizal roots. The previously described mycorrhiza-specific phosphate transporter gene, OsPT11, and plasma membrane H⁺-ATPase gene, OsHA1, that were used as positive controls for the mycorrhiza-specific accumulation of transcripts, were strongly upregulated in Applicants transcriptome of rice mycorrhizal roots. Quantitative reverse transcription-polymerase chain reaction (RT-PCR; qRT-PCR) analysis of one of the two RNA preparations used for RNA-seq, validated the transcriptome results regarding the mycorrhiza-inducible nature of all N transport- and metabolism-related DEGs, and confirmed that OsNPF4.5 was the strongest up-regulated putative nitrate transport gene, with the transcripts increased by over 500 folds in mycorrhizal roots relative to the mock control (FIG. 11D). These findings suggest the presence of a symbiotic pathway for nitrate uptake in the mycorrhizal rice plants.

### Example 2.3. AM Fungal Colonization Promotes Rice Growth and Nitrate Uptake

To investigate the potential role of AM symbiosis in plant nitrate acquisition, rice plants were grown in a sand/soil mixture-based substrate, inoculated or mock-inoculated with AM fungus (R. irregularis) and supplemented with 0.25, 1.0, 2.5 and 5.0 mM of NO₃⁻ as N sources. After 8 weeks of growth, all mycorrhizal rice plants supplied with NO₃⁻ showed a statistically significant increase in root and shoot biomass and N and P accumulation in both shoots and roots compared with nonmycorrhizal plants, except those supplied with 0.25 mM NO₃⁻ that did not differ significantly in biomass with the mock-inoculated control plants.

Applicants' findings highlight that AM fungal colonization could promote rice plant growth and nitrate uptake. The lack of growth promotion observed in mycorrhizal rice plants supplemented with 0.25 mM NO₃⁻ might be partially ascribed to a relatively smaller shoot N increment, and lower colonization efficiency and arbuscule incidence, compared with those grown under high NO₃⁻. It has been documented that the mycelium of AM fungi constitutes considerable N sink, and competition for N would potentially reduce N delivery and mycorrhizal benefits to the host plant under N-limited conditions, which may in turn lead to a negative effect on AM fungal colonization.

The reduced mycorrhization in low-NO₃⁻-treated plants was confirmed by a decreased expression of the AM-specific marker gene OsPT11. The reduced colonization efficiency caused by low NO₃⁻ application was also observed in mycorrhizal sorghum plants. In contrast to high phosphate that is well known to inhibit the symbiotic process, Applicants found that high NO₃⁻ concentrations (5 mM) did not inhibit mycorrhization. These results suggest that phosphate but not nitrogen availability is the major signal that the plants perceive to activate or repress the AM symbiosis.

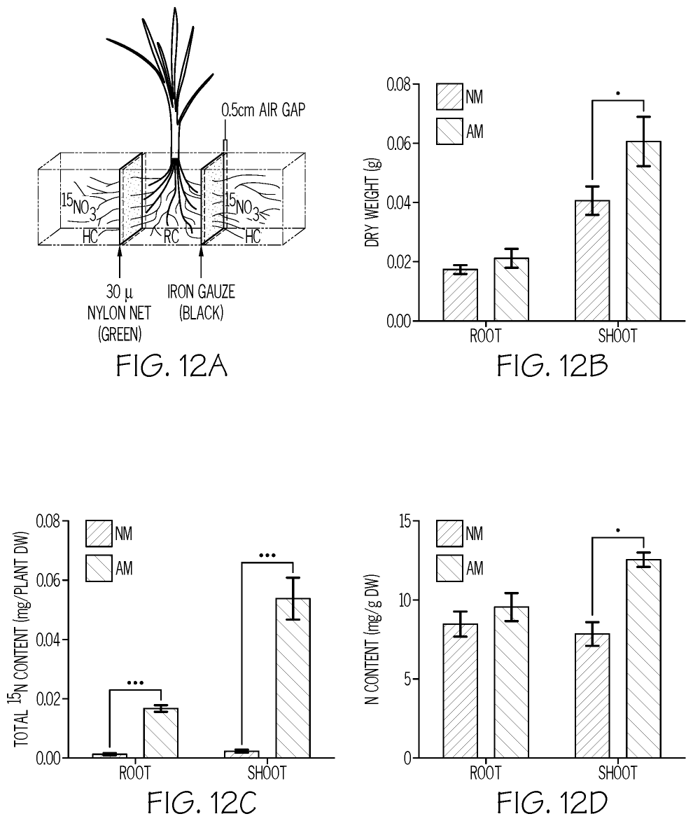

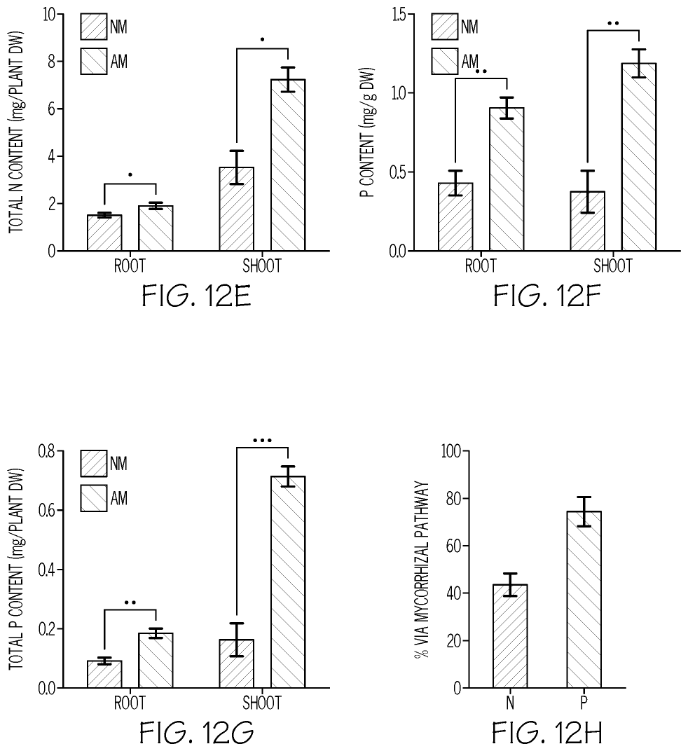

To further evaluate the contribution of symbiotic NO₃⁻ uptake to the overall N nutrition of the mycorrhizal rice plants, ¹⁵NO₃⁻-labeled uptake measurement was performed using a compartmented growth system (FIG. 12A) containing a middle root/fungal compartment (RFC) that was separated by two 30-mm nylon meshes from two hyphal compartments (HCs) with a 0.5 cm air gap between the RFC and HC compartments to prevent ¹⁵NO₃⁻ diffusion (see diagram in FIG. 12A). Control and R. irregularis-inoculated rice seedlings were grown in the RFC compartment supplemented with 2.5 mM NO₃⁻ as sole N source, and an equal amount of ¹⁵NO₃⁻ was provided to the two HC compartments. ¹⁵N, total N, and total P contents were determined in both roots and shoots of mock and mycorrhizal rice plants at 6-weeks post inoculation (wpi). Mycorrhizal plants showed an increase of 49±15% in shoot biomass (dry weight) compared with the nonmycorrhizal controls (FIG. 12B). High ¹⁵N accumulation was readily detectable in the roots and shoots of inoculated plants, but barely detectable in all the mock-inoculated plants (FIG. 12C), indicating that fungal hyphae could reach and take up nutrients from HCs and that no NO₃⁻ diffusion across the nylon meshes occurred.

Mycorrhizal plants also showed an increase of 60±8% in shoot N content and a 106±15% in total shoot N content per plant as compared to the controls (FIGS. 12D and E). Applicants also found that mycorrhizal plants had a three-fold increase in shoot P content and a 5-fold increase in total shoot P content per plant over the control (FIGS. 12F and G). In contrast to P content that was significantly increased in the root of mycorrhizal plants, N content in the root did not differ significantly between mycorrhizal plants and mock-inoculated plants (FIG. 12D-G), suggesting a more rapid transport of N than P from root to shoot in mycorrhizal plants. A determination of the percentage of N and P transferred via the mycorrhizal pathway showed that 42±4% N and 74±7% P was taken up via the mycorrhizal pathway (FIG. 12H).

Applicants' results on P uptake via the symbiotic pathway are similar to that of a previous report demonstrating that mycorrhizal rice received over 70% of its Pi via the symbiotic uptake pathway, suggesting that Applicants' experimental set up is adequate to measure the contribution of the mycorrhizal route on nutrient uptake. These findings highlight that in addition to the mycorrhizal P uptake pathway, rice also activates an efficient route for symbiotic N acquisition upon the formation of AM symbiosis.

### Example 2.4. Identification and Characterization of the AM-Induced OsNPF4.5 in Rice

The increased nitrate uptake of mycorrhizal rice plants prompted Applicants to speculate that AM-induced nitrate transporter(s) might be required for nitrate uptake at the symbiotic interface. Since OsNPF4.5 is the gene encoding a putative nitrate transporter of the NRT1/NPF family with the highest upregulated expression in mycorrhizal roots, Applicants decided to further investigate its expression pattern and possible function. An attempt to clone the full-length open reading frame (ORF) of OsNPF4.5 based on the predicted online information (0s01g0748950/LOC_Os01g54515.1) was unsuccessful. Thus, RNA-based RACE-PCR (First Choice RLM-RACE Kit, Ambion) was employed to obtain a full-length cDNA of OsNPF4.5.

By comparing the cDNA and its genomic DNA sequences, OsNPF4.5 was found to contain an 1830 bp-length ORF separated by 6 introns. As most known plant NPF transporters, OsNPF4.5 putatively harbors 12 trans-membrane domains with an intracellular central loop. Phylogenetic analysis grouped OsNPF4.5 and its orthologues together with several NPF homologues that have been evidenced to possess nitrate transport capacity, such as the rice OsNPF6.3 and OsNPF6.5. Overall comparison of the crystal structure of the well-known nitrate transporter AtNRT1.1/CHL1 and the model structure of OsNPF4.5, revealed a high level of superposition between the two protein structures.

The model structure of OsNPF4.5 suggest the presence of 12 transmembrane helices disposed in a similar orientation as those of AtNRT1.1 forming the NO₃⁻ transport tunnel, in which some important residues such as L49, V53, and K164, and the phosphorylation site T101 in AtNRT1.1 are also conserved in OsNPF4.5. A sequence alignment of AtNRT1.1, OsNRT1.1, OsNPF4.5, and multiple OsNPF4.5 orthologues from diverse monocot and dicot plant species, and secondary structure assignment according the OsNPF4.5 model and the AtNRT1.1 reported structure, showed that the 12 putatively transmembrane helices and the residues mentioned above are also highly conserved in OsNRT1.1, the rice orthologue of AtNRT1.1, and in the different OsNPF4.5 orthologues. However, some other residues forming part of the transport tunnel and the binding pocket in OsNPF4.5, are different from those present in equivalent positions in AtNRT1.1 and OsNRT1.1, such as L373, Q377, D499, Y534 (in reference to OsNPF4.5 residues position), but highly conserved among NPF4.5 orthologues.

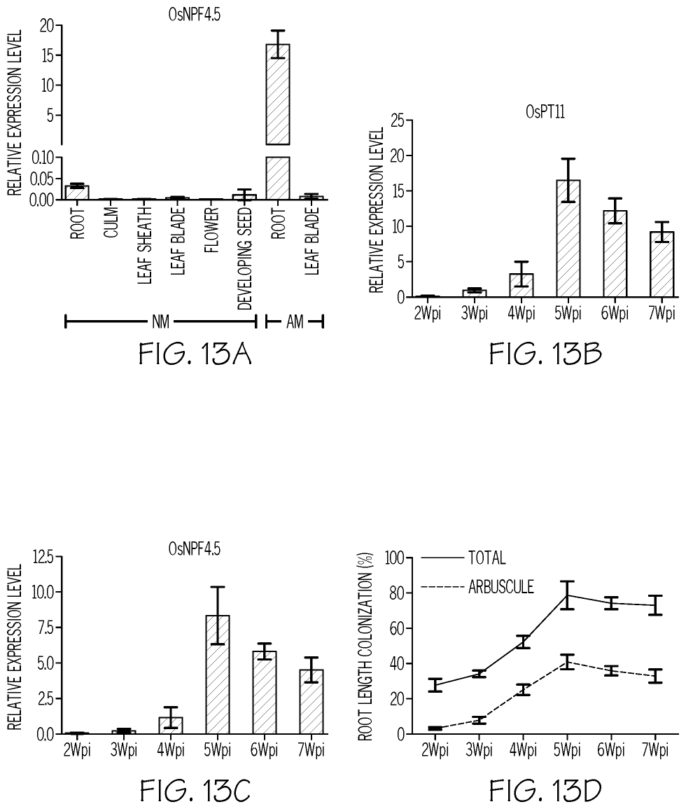

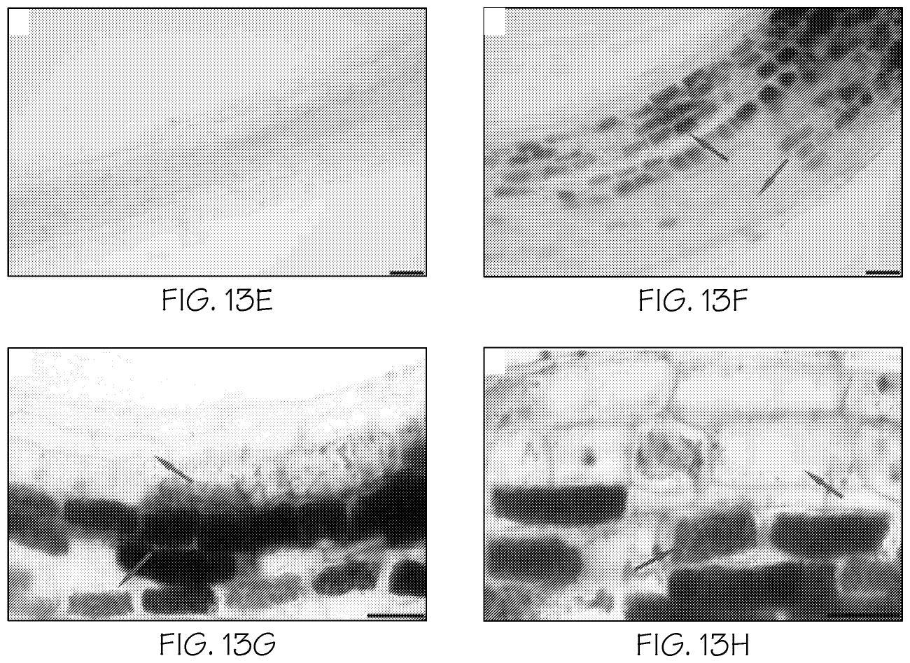

Besides mycorrhizal roots, OsNPF4.5 transcripts were barely detectable in other tissues, including culm, leaf sheath and blade, flower, and developing seeds (FIG. 13A). Unlike the known nitrate transporters, such as OsNPF6.3/NRT1.1A and OsNPF6.5/NRT1.1B, having an inducible expression in response to NO₃⁻, or even NH₄⁺ supply, OsNPF4.5 showed no conspicuous response to external NO₃⁻ or NH₄⁺ application or deprivation. A time-course expression analysis further revealed similar kinetics of transcript accumulation between OsNPF4.5 and OsPT11 in rice mycorrhizal roots, with expression starting to be detected 3 wpi and reaching a maximum 5 wpi in both cases (FIGS. 13B and C).

The kinetic of expression of OsNPF4.5 and OsPT11 also correlated well with mycorrhizal colonization intensity (FIGS. 13B-D). To explore in more detail the expression pattern of OsNPF4.5, Applicants constructed a transcriptional fusion between the promoter of this nitrate transporter and the coding sequence of the GUS reporter gene. Histochemical GUS assays confirmed that OsNPF4.5 expression was practically undetectable in non-mycorrhizal roots (FIG. 13E), whereas intense GUS staining was detected in mycorrhizal roots (FIGS. 13F and 13G). Co-localization of GUS expression and AM fungal structure by overlay of Magenta-GUS with Trypan Blue staining showed that the GUS activity driven by the OsNPF4.5 promoter was exclusively confined to cells containing arbuscules (FIG. 13H). Subcellular localization analysis showed that the eGFP-OsNPF4.5 fusion protein expressed under control of the 35S cauliflower mosaic virus promoter in N. benthamiana epidermal cells, was exclusively localized to the plasma membrane.

Expression of the OsNPF4.5-eGFP fusion protein from its own promoter in mycorrhizal rice showed a distinct localization signal, likely the PAM, in arbuscule-containing cells. These results confirm that the expression of OsNPF4.5 is specific in arbuscule-containing cells and that OsNPF4.5 is a membrane-localized protein probably present in the PAM upon AM symbiosis.

To determine whether the NPF4.5 orthologues in other mycorrhizal plant species were also inducible in response to AM symbiosis, Applicants quantitatively assayed the expression of the NPF4.5 orthologues in Medicago (MtNPF4.5), maize (ZmNPF4.5) and sorghum (SbNPF4.5). Applicants' results showed that expression of all these three NPF4.5 orthologues was barely detectable in roots of non-AMF inoculated roots. By contrast, AMF inoculation strongly induced expression of ZmNPF4.5 in maize, SbNPF4.5 in sorghum, while MtNPF4.5 was slightly induced in Medicago. The strong inducibility of SbNPF4.5 transcripts in response to AM symbiosis was confirmed in the RNASeq data from a recent report on the global transcriptional changes induced by arbuscular mycorrhizal fungi on several Sorghum bicolor accessions.

These results suggest the likely presence of a conserved symbiotic NO₃⁻ uptake route at least in gramineous species. It was previously reported that symbiosis with R. irregularis strongly induced the expression of the OsNPF4.5 orthologues in Populus trichocarpa (POPTR_004g064100) and Helianthus annuus (HanXRQChr15g0472261), suggesting that NPF4.5 could play an important role in symbiotic NO₃⁻ nutrition in plants outside gramineae.

### Example 2.5. OsNPF4.5 Possesses Nitrate Transport Capacity In Vitro and In Vivo

The NO₃⁻ transport capacity of OsNPF4.5 was initially evaluated by heterologous expression in Xenopus oocytes. CHL1/AtNRT1.1, the well-established dual-affinity NO₃⁻ transporter, was used as a positive control. Assays of ¹⁵N-nitrate uptake showed that the NO₃⁻ uptake was much higher in oocytes injected with CHL1 complementary RNA (cRNA) than in those water-injected controls under both low (0.25 mM) and high (10 mM) NO₃⁻ concentrations. Oocytes injected with OsNPF4.5 cRNA and incubated in 0.25 mM NO₃⁻ showed no significant difference in nitrate uptake activity than the water-injected controls, while those incubated in 10 mM NO₃⁻ showed a 2-fold increase in NO₃⁻ uptake when compared with the water-injected oocytes at pH 5.5 (FIG. 14A), but not at the pH 7.4 (FIG. 14B).

The Kₘ of OsNPF4.5 affinity for NO₃⁻ uptake was calculated from the net NO₃⁻ accumulation of the oocytes incubated in a series of concentrations (0.25, 1, 2.5, 5, 10, 15, and 20 mM) of ¹⁵N—NO₃⁻, and was estimated as 1.95±0.48 mM (FIG. 14C). Inward currents responding to alterations in membrane potential could also be evoked by 10 mM NO₃⁻ supply for OsNPF4.5-injected oocytes (FIG. 14D). These results demonstrate that OsNPF4.5 functions as a low-affinity, pH-dependent NO₃⁻ transporter when expressed in Xenopus oocytes.

To assess whether overexpression of OsNPF4.5 can facilitate NO₃⁻ uptake in vivo, Applicants generated transgenic rice plants constitutively overexpressing OsNPF4.5 under the control of a maize ubiquitin promoter and performed both short-term and long-term hydroponic uptake experiments. In the short-term uptake experiment, wild-type (WT) control plants and five independent OsNPF4.5-overexpressing transgenic lines, referred as OX lines, were subjected to N deprivation for 4 days, and then resupplied with 2.5 mM ¹⁵N-labeled NO₃⁻ or NH₄⁺ for 10 minutes. When supplied with 2.5 mM ¹⁵NO₃⁻, all the OX lines showed a 24% to 50% higher ¹⁵N uptake than WT plants (FIG. 14E). By contrast, no difference in ¹⁵N accumulation could be observed between the WT and OX plants when supplied with ¹⁵NH₄⁺ supply (FIG. 14F).

For the long-term uptake experiment, seedlings of WT plants and three OX lines, were subjected to N deprivation for 4 days, and then resupplied with 2.5 mM NO₃⁻ or NH₄⁺ for 3 weeks. When supplemented with 2.5 mM NO₃⁻, OX transgenic lines showed a 25% to 46% increase in shoot biomass, a 6 to 8-fold increase in NO₃⁻ content in roots, a 2 to 3-fold increase in NO₃⁻ content in shoots and an increase of 80 to 110% in total N content in both shoot and root when compared to WT plants. The high NO₃⁻ and total N content phenotype of OX plants seems to be due to the high level of OsNPF4.5 transcripts in OX transgenic rice lines as it was increased thousands of folds compared to that in WT plants. In the long-term uptake experiment no significant difference in either plant biomass or total N content could be observed between the WT and OX transgenic plants supplied with NH₄±. In OsNPF4.5-overexpressing rice plants supplied with NO₃⁻, increased expression of some N assimilation-related genes such as OsNR1/2 and OsGS1 was observed. All these results lend solid evidence to support that OsNPF4.5 has NO₃⁻, but not NH₄⁺ transport capacity. The significantly superior capacity of OX plants in NO₃⁻ uptake opens the possibility of using OsNPF4.5 in breeding programs to improve rice N use efficiency, as had been proposed for several other NO₃⁻ transporter genes.

### Example 2.6. Loss of OsNPF4.5 Function Decreases Symbiotic Nitrate Transport and Arbuscule Incidence

The mycorrhiza-specific property of OsNPF4.5 inspired Applicants to investigate whether OsNPF4.5 contributes to the symbiotic NO₃⁻ uptake and/or AM formation. To test this, osnpf4.5 knockout mutants were generated with the CRISPR-Cas9 system using three different spacers targeting the coding sequence of OsNPF4.5. Two out of the three spacers worked effectively in the editing system resulting in the generation of nine mutant lines which were screened by PCR sequencing, and three independent homozygous lines were used for further study. Osnpf4.5-1 contains a “T” insertion at nucleotide 483 of the ORF that causes a shift in reading frame, and osnpf4.5-2 harbor a “G” deletion at position 482 and osnpf4.5-3 an “A” deletion at position 708. In all cases CRISPR-Cas9 mutations resulted in frame shifts and premature termination in the first half of OsNPF4.5. No significant difference in N accumulation could be observed between the three osnpf4.5 mutants and WT plants grown under hydroponic conditions supplied with either 2.5 mM NO₃⁻ or NH₄⁺ as a N source, or a sand-based pot culture supplied with 2.5 mM NO₃⁻ in the absence of AM fungal inoculation.

When inoculated with R. irregularis, the mycorrhizal WT plants increased shoot biomass and shoot N content by 31±6% and 39±7%, respectively, relative to non-inoculated plants, whereas osnpf4.5 plants showed only a 10±4% increase in shoot biomass and no significant increase in shoot N content as compared to non-inoculated WT or mutant lines. When total N and P content was quantified, Applicants found that inoculated WT plants increased 65±6% and 275±19% in total N and P content, respectively, compared to non-inoculated WT plants. By contrast, osnpf4.5 plants displayed an increase of 28% to 34% and 234% to 247% in total shoot N and P content relative to that determined in mock-inoculated WT and mutant lines. These results strongly suggest that OsNPF4.5 plays an important role in the mycorrhizal NO₃⁻ uptake pathway, but not in the direct uptake pathway. Moreover, the reduction in the growth promotion of inoculated osnpf4.5 mutants is most probably due to a reduction in N-supply because of the lack of a functional OsNPF4.5 transporter. However, Applicants could not rule out that the reduction in growth promotion in inoculated osnpf4.5 plants might be partially caused by a colonization difference between the WT and osnpf4.5 plants.

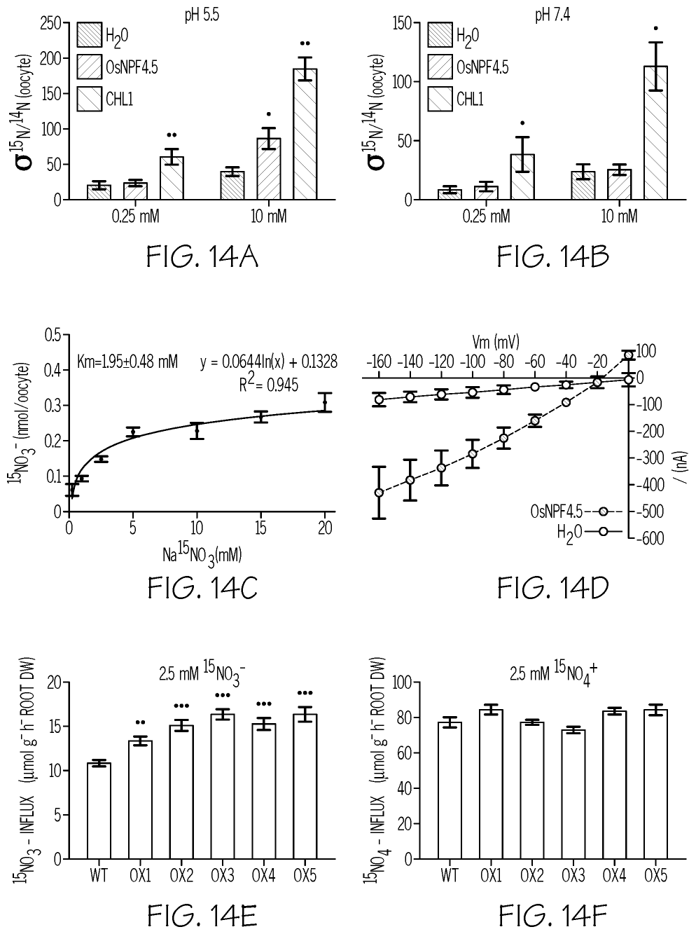

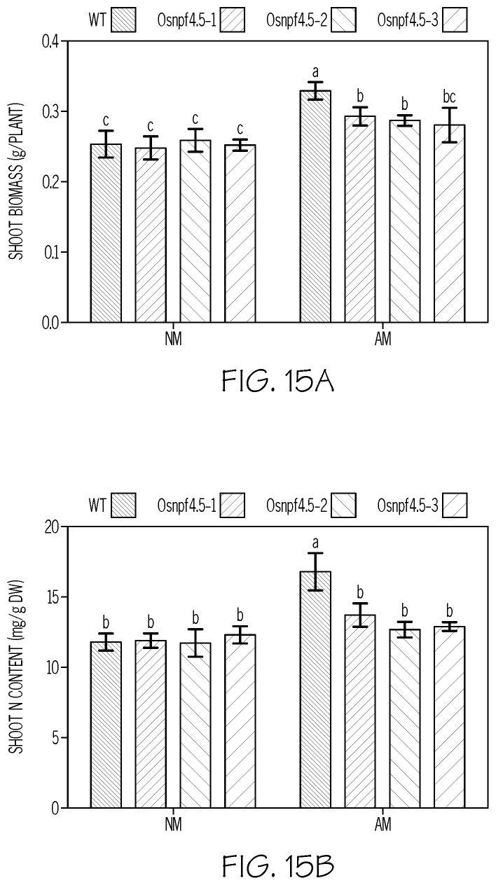

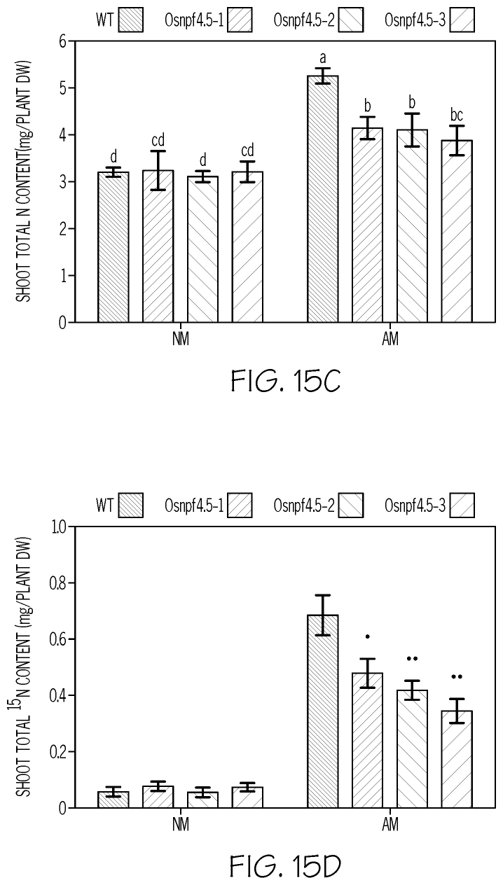

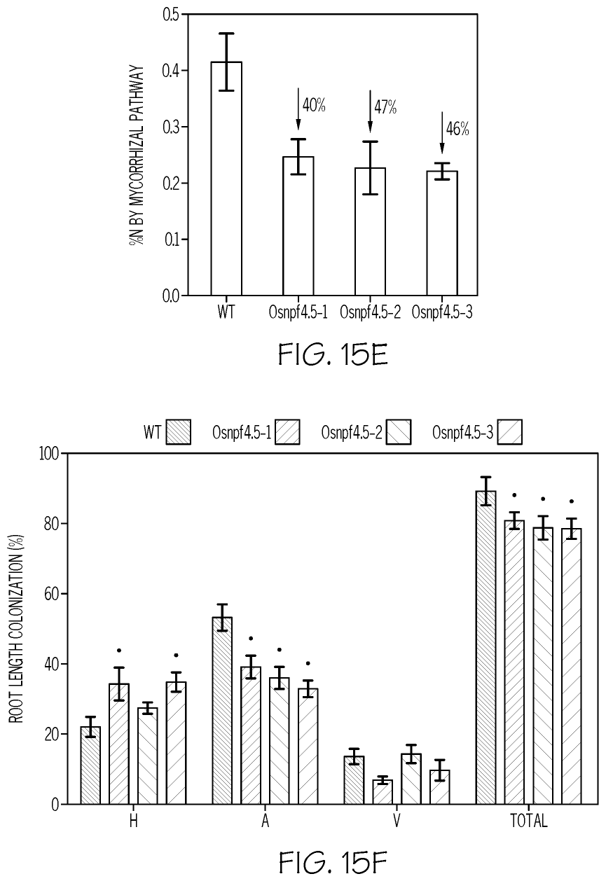

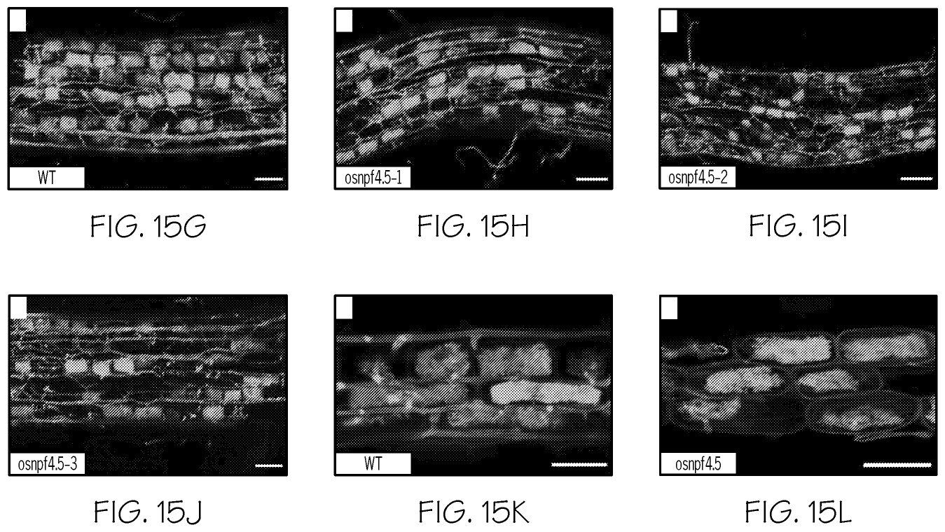

To quantify the potential contribution of OsNPF4.5 to mycorrhizal NO₃⁻ uptake, seedlings of WT and osnpf4.5 plants were cultivated in the compartmented growth system, and 2.5 mM NO₃⁻ and ¹⁵NO₃⁻ were supplied to the RFC compartment and HC compartments, respectively (FIG. 12A). Consistent with the results obtained from the pot culture, inoculated WT plants increased shoot biomass by about 30±4%, shoot N content by about 42±5% and total N content by 64±5% relative to mock-inoculated WT (FIG. 15A-C).

By contrast, mycorrhizal osnpf4.5 mutant plants showed an increase of only 15±4% in shoot biomass and no difference in shoot N content relative to mock-inoculated WT and the respective mutant lines (FIGS. 15A-C). Both the WT and osnpf4.5 mycorrhizal plants contained a higher ¹⁵N than the corresponding mock-inoculated control plants (FIG. 15D), indicating that both the WT and osnpf4.5 can take up NO₃⁻ from hyphal compartments via the fungal hyphae. However, the significant decrease in ¹⁵N accumulation observed in the shoots of osnpf4.5 mycorrhizal plants compared with that in the mycorrhizal WT plants highlights the important role of OsNPF4.5 in mycorrhizal NO₃⁻ uptake.

Mutation of OsNPF4.5 led to a decrease of the percentage of mycorrhizal N uptake contribution from 42% in WT plants to less than 25% in osnpf4.5 mutant lines (FIG. 15E), indicating that OsNPF4.5 may account for approximately 45% of the mycorrhizal N uptake when supplied with NO₃⁻ as N sources. Since Applicants have solid evidence showing that OsNPF4.5 has NO₃⁻ transporter activity, Applicants propose that NO₃⁻ is the molecule that is released into the periarbuscular space and imported by root cells using NPF4.5 and other nitrate transporters. However, since some NO₃⁻ transporters have also been shown to be able to transport amino acids and small peptides, Applicants cannot exclude the possibility that at least a fraction of the symbiotic N is supplied to the plant in the form of organic N molecules.

The bidirectional nutrient exchange between host plants and AM fungi is thought to follow a “free-market” model, in which both symbionts can exert control over their partners. A mutually stimulating mechanism has been repeatedly proposed during the simultaneous exchange of C and Pi between the two partners. Blocking mycorrhizal P transport via silencing the Pi transporters or H⁺-ATPases located in the PAM caused a remarkable defect in mycorrhization and arbuscule development. To determine whether alteration in symbiotic nitrate transport caused by mutation of OsNPF4.5 affects AM symbiosis, the degree of AM colonization, as well as the arbuscule morphology and populations in the mycorrhizal roots of WT and osnpf4.5 mutant lines were assessed 6 wpi (FIGS. 15F-L).

Compared to WT plants, a small but statistically significant decrease of approximately 10% in total root length colonization and nearly 20% in arbuscule colonization rate was observed in osnpf4.5 mutant lines (FIG. 15F-J). It is worth noting that although reduced in arbuscule colonization rate, well-developed arbuscules were clearly observed in osnpf4.5 plants (FIG. 15K, L), suggesting that symbiotic NO₃⁻ transport might not be an essential requirement for arbuscule development.

### Example 2.7. Conclusion

NH₄⁺ and NO₃⁻ are the two inorganic forms of N taken up by plants. Previous studies in several plant species have suggested the presence of a symbiotic NH₄⁺/NH₃ transport route via the interfacial apoplast into plant root cells probably mediated by the AM-induced plant NH₄⁺ transporters located on the PAM. Rice is thought to have evolved a high-efficiency NH₄⁺ transport system, as in paddy fields NH₄⁺ is the major N source. RNA sequencing analysis in this Example, however, allowed to identify multiple genes involved in nitrate transport and metabolism, but only one NH₄⁺ transporter gene that were significantly upregulated in rice mycorrhizal roots (FIG. 11C).

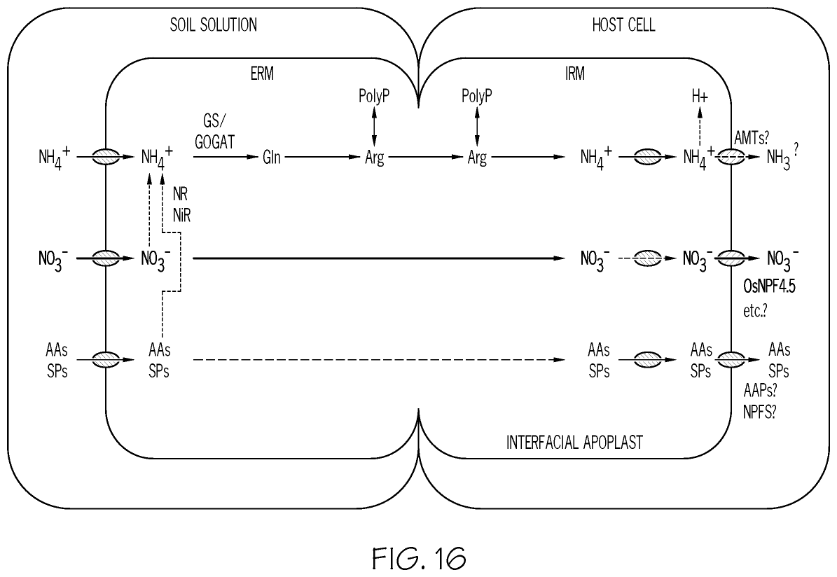

Applicants' findings obtained from the compartmented culture system enrich the previously proposed mycorrhizal N uptake model by clearly indicating the presence of a symbiotic NO₃⁻ acquisition route (FIG. 16) from NO₃⁻ uptake by extraradical mycelium to NO₃⁻ translocation at the fungus-root interface mediated by plant NO₃⁻ transporters (FIG. 16).

Applicants show that mycorrhizal NO₃⁻ uptake route could contribute up to 42% of the overall rice N uptake, when NO₃⁻ was supplied as N source. Moreover, Applicants' results demonstrate that about 45% of the mycorrhizal NO₃⁻ was delivered via OsNPF4.5, the strongest AM-induced NO₃⁻ transporter. Given that several NPF homologues in diverse plant species have been shown to have the ability to transport dipeptides and amino acids, as well as other substrates, Applicants cannot completely exclude the possibility that in addition to NO₃⁻, OsNPF4.5 might also have the ability to transport other organic N substrates, such as small peptides and amino acids.

Applicants' results suggest that AM symbiosis not only activates the transport of NO₃⁻ but also N assimilation in general because genes encoding nitrate reductase, nitrite reductase, glutamine synthetase, and glutamate synthase are also upregulated during mycorrhization with R. irregularis. In this Example, Applicants also revealed a high conservation in both the secondary structure and residues potentially involved in NO₃⁻ binding and transport among rice OsNPF4.5 and its orthologues from other dicot and monocot plant species. The strong induction of the orthologues, ZmNPF4.5 and SbNPF4.5 observed in maize and sorghum, respectively, in response to AM symbiosis suggests that the NPF4.5-mediated symbiotic NO₃⁻ uptake route as an important pathway for mycorrhizal N acquisition might be highly conserved at least in gramineous species.

### Example 2.8. Plant Materials and Growth Conditions

The rice (Oryza sativa ssp japonica) wild-type and transgenic plants used in this Example were in the cv Nipponbare background. Rice seeds were surface sterilized and germinated in a growth chamber programmed for 14-h light at 28° C. and 10-h dark at 22° C. and maintained to grow in one-half IRRI nutrient solution for one week. Seedlings produced as mentioned above were then transferred to pot or compartmented culture inoculation with AM fungus. For pot culture, eight seedlings of WT or each line of individual mutants were transplanted to four holes (two seedlings as a replicate were placed into each hole) in a pot (35 cm diameter×24 cm height) filled with a 4:1 mixture of sterilized sand and low-N soil (the soil contains 2.2 mg kg⁻¹ NH_{4+, 3.7} mg kg⁻¹ NO₃⁻, and 1.4 mg kg⁻¹ available P). The seedlings in each hole were inoculated with approximately 200 Rhizophagus irregularis spores around the roots. The nonmycorrhizal control plants were obtained by inoculation with autoclaved inoculum. The plants in each pot were regularly watered and fertilized weekly with 500 ml nutrient solution containing 2.5 mM NO₃⁻ (or other concentrations for different treatments), and 30 μM Pi, as well as the other essential nutrients from the modified IRRI nutrient solution recipe.

### Example 2.9. Determination of Mycorrhizal Nitrate Uptake Contribution

A compartmented culture system was employed to investigate the contribution of symbiotic NO₃⁻ uptake to the overall N nutrition of mycorrhizal rice WT and osnpf4.5 mutant plants (FIG. 12A). The culture system contains a middle root/fungal compartment (RFC) and two hyphal compartments (HCs) (each compartment is 10×10×12 cm in length, width and height). All three compartments were filled with approximately 1 L sand/low-N soil mixture. Two seedlings of WT or mutant plants were grown in the RFC inoculated with R. irregularis or autoclaved inoculum (as control) for 6 weeks. Each treatment included 5 compartmented boxes as independent biological replicates. The plants in RFC were regularly watered and fertilized weekly with 250 ml nutrient solution containing 2.5 mM NO₃⁻ as the N source, and simultaneously the two HCs were supplied with equal amount of nutrient solution containing 2.5 mM ¹⁵NO₃⁻. To monitor whether fungal hyphae could reach and take up NO₃⁻ from HCs, the ¹⁵N content in the inoculated and mock-inoculated plants was determined. To assay ¹⁵N content, harvested plants were rinsed for 1 min in 0.1 mM CaSO4 solution and then roots and shoots were separated. The collected root and shoot samples were dried at 70° C. and weighted before being ground. One mg of the finely ground powder for each sample was used to determine the ¹⁵N content by an isotope ratio mass spectrometer with an elemental analyzer (DELTA V ADVANTAGE isotope Ratio MS, Thermo Fisher). Total shoot N, root N or ¹⁵N content (mg/plant)=shoot N, root N or ¹⁵N content (mg/g)×shoot or root biomass (g, dry weight). Total N content in the plant=total shoot N content+total root N content. The percentage of contribution of the mycorrhizal pathway to total N uptake in WT or osnpf4.5 mutants was calculated with the formula [(Total N content in AM plant−Total N content in NM plant)/Total N content in AM plant]×100%. The contribution of OsNPF4.5 to mycorrhizal pathway of NO₃⁻ uptake was calculated with the formula [(mycorrhizal N uptake contribution in WT plants−mycorrhizal N uptake contribution in osnpf4.5 mutants/mycorrhizal N uptake contribution in WT plants]×100%.

### Example 2.10. RNA Sequencing

The inoculated and mock-inoculated seedlings were irrigated with IRRI nutrient solution containing 1.25 mM NH₄NO₃ and 30 μM Pi weekly. The roots of the mycorrhizal and nonmycorrhizal plants were collected 6 weeks post inoculation. Total RNA was isolated using the RNEasy Plant Maxi kit (Qiagen, Hilden, Germany). Three biological replicates for each treatment were used for RNA sequencing reaction performed on an Illumina Hiseq 2500. After trimming and eliminating low quality reads, 39463820 and 38621548 clean reads were obtained for inoculated and control plants, respectively, which accounted for over 95% of the total sequences. The transcriptome data analysis was commercially conducted by the CapitalBio Corporation (Beijing, China).

### Example 2.11. RNA-Based RACE PCR

The full-length cDNA of OsNPF4.5 was obtained by rapid amplification of cDNA ends (RACE) (First Choice RLM-RACE Kit, Ambion). One and ten μg of total RNA were used for the 3′ and 5′ RLM-RACE protocols, respectively, following the manufacturer's instructions strictly. The specific primers used for amplifying the 5′ and 3′ ends of OsNPF4.5 cDNA are: 5′ outer primer, ggccaatgaaagtgtccgcgaag (SEQ ID NO: 10), 5′ inner primer, acggctagagacaacgaggcaagg (SEQ ID NO: 11), 3′ outer primer, gccgcagttcaccgtgtt (SEQ ID NO: 12), and 3′ inner primer, tcatcgggctcctcgagtt (SEQ ID NO: 13).

### Example 2.12. Phylogenetic Analysis

The unrooted phylogenetic tree of the plant NPF homologues was constructed using their protein sequences by the Neighbor-Joining algorithm within the MEGA 6 program with bootstrapping value (range 0-100). For tree construction Applicants used the OsNPF4.5 orthologues in Medicago (MtNPF4.5), maize (ZmNPF4.5) and sorghum (SbNPF4.5) as previously identified by others and confirmed by bidirectional BLAST analysis, and other nitrate transporters. The reference numbers of the protein sequences used for constructing the tree are the following: OsNPF1.3, XP_015636060.1; OsNPF5.4, XP_015612792.1; OsNPF8.3, XP_015634046.1; LjNPF8.6, IPR000109; MtNPF1.7, XP_003588616.1; MtNPF6.8, XP_003616931.1; OsNPF6.3 (OsNRT1.1A), XP_015650127.1; OsNPF6.5 (OsNRT1.1B), XP_015614015.1; OsNPF6.4 (OsNRT1.1C), XP_015632236.1; OsNPF2.4, XP_015630690.1; OsNPF2.2 (OsPTR2), XP_015620477.1; OsNPF7.2, XP_015627752.1; ZmNPF6.6, XP_008658424.1; ZmNPF6.4, NP_001145735.1; AtNPF6.4 (AtNRT1.1), NP_563899.1; AtNPF4.6 (AtNRT1.2), NP_564978.1; AtNPF5.12 (AtTOB1), NP_177359.1; AtNPF6.2 (AtNRT1.4), NP_850084.1; AtNPF5.5, NP_181345.1; AtNPF1.1 (AtNRT1.12), NP_188239.1; AtNPF6.4 (AtNRT1.3), NP_188804.1; AtNPF4.1 (AtNIT3), NP_189163.1; AtNPF4.2 (AtNIT4), NP_189165.1; AtNPF2.7 (AtNAXT1), NP_190151.1; AtNPF2.3, NP_190154.1; AtNPF2.10 (AtGTR1), NP_566896.2; AtNPF7.2 (AtNRT1.8), NP_193899.2; AtNPF2.9 (AtNRT1.9), NP_173322.1; AtNPF5.10, NP_173670.2; AtNPF4.5 (AtAIT2), NP_973919.1; AtNPF2.12 (AtNRT1.6), NP_174028.2; AtNPF7.3 (AtNRT1.5), NP_174523.2; AtNPF1.2 (AtNRT1.11), NP_175630.1; AtNPF8.5 (AtPTR6), NP_176411.2; AtNPF3.1 (AtNitr1), NP_177024.1; OsNPF7.3 (OsPTR6), XP_015633790.1; ZmNPF4.5, XP_020406064.1; SbNPF4.5, XP_021311980.1; SbNPF1.2, XP_002458530.1; GmNPF4.5, XP_003532772.2; MtNPF4.5, XP_024627880.1.

### Example 2.13. Vectors, Strains and Rice Gene Transformation

For promoter-GUS assays, a 2030-bp promoter fragment of OsNPF4.5 immediately upstream of the translation start ATG was amplified and inserted into the pCAMBIA1300 binary vector to replace the CaMV35S promoter in front of the GUS reporter gene. To construct the OsNPF4.5 overexpression vector, the coding sequence of OsNPF4.5 was amplified and cloned into the binary vector pTCK303 under the control of a maize ubiquitin promoter using the ClonExpress II One Step Cloning Kit (Vazyme Biotech, Nanjing, China). The CRISPR/Cas9 gene knockout constructs were generated using the pH-Ubi-cas9-7 vector. Three different spacers (spacer1, ggggaagacctgcaataaga (SEQ ID NO: 14), spacer2, gttcgaccccaagtgcgaga (SEQ ID NO: 15), and spacer3, gtgtggatccagagctacaa (SEQ ID NO: 16)) targeting the coding sequence of OsNPF4.5 were selected from the rice-gene-specific spacers library. These spacers were firstly cloned into the intermediate vector pOs-sgRNA via BsaI, and then introduced into the expression vector pH-Ubi-cas9-7 using the Gateway recombination technology (Invitrogen). All the resulting constructs were transformed into the Agrobacterium tumefaciens EHA105 strain. The transformation of rice plants was carried out. The screening of mutant lines was performed by PCR sequencing. Except spacer1 that did not work effectively in the editing system, the other two spacers successfully resulted in the generation of several homozygous mutant rice lines.

### Example. 2.14. Subcellular Localization Analysis

The CDS of OsNPF4.5 was fused in frame with eGFP via cloning into the binary vector pRCS2-ocs-nptII. The resulting vector, named 35S::eGFP-OsNPF4.5, were transformed into the EHA105 strain. The agroinfiltration of tobacco leaves and the imaging of eGFP fluorescence were performed. For assaying the subcellular localization of OsNPF4.5 in mycorrhizal roots, the native promoter of OsNPF4.5 was amplified and inserted into the pCAMBIA1300 vector to replace the CaMV35S promoter, and then the OsNPF4.5-eGFP chimeric gene was cloned and inserted into the vector under the control of the OsNPF4.5 promoter. The resulting vector, named NPF4.5ₚᵣₒ::OsNPF4.5-eGFP, was introduced into the EHA105 strain, and used for transformation of rice. The transgenic plants were then transferred to sand-based pot culture for inoculation with the AM fungus R. irregularis. The eGFP image was observed with a confocal microscope (Leica Confocal TCS-SP8) 6 weeks post inoculation.

### Example 2.15. Mycorrhizal Colonization Quantification

Histochemical staining of the GUS activity in transgenic plants was performed. Mycorrhizal colonization was quantified based on the grid line intersect method using a binocular microscope (Leica, Germany). The measurement of arbuscule sizes in the arbuscule populations was performed. To visualize the fungus, roots were stained in 0.2 mg/ml WGA Alexafluor 488 solution. For assessment of arbuscule populations, the stained root segments were observed using the confocal microscope, and arbuscules were grouped into three size classes (0-30 μm, 30-50 μm, and >50 μm) based on their lengths and the percentage of arbuscules in each size class was counted. Arbuscule size was determined by measuring the length of all the visible arbuscules (at least 200 arbuscules) in five to ten independent infection units for each root sample, and the average and the SE of each arbuscule size are graphed from three independent biological replicates.

### Example 2.16. Determination of N and P Contents

The digestion of dried plant material with 98% H₂O₂ and 30% H₂O₂ and the assay of total P content with the molybdate blue method were performed. The assay of total N and nitrate was performed.

### Example 2.17. Analysis of Gene Expression

RNAs were extracted by using TRIzol reagent (Invitrogen). Two micrograms of total RNA were used for RT-PCR reactions using MLV reverse transcription kit (TaKaRa). Quantitative RT-PCR was performed based on the instructions of the SYBER premix ExTaq kit (TaKaRa) on an Applied Biosystems Plus Real-Time PCR System by using gene-specific primers. The expression of Os-Actin (0503g50885) was used for normalization. Four biological replications were performed.

### Example. 2.18. ¹⁵N-Nitrate Uptake Assay in Xenopus laevis Oocytes

The CDS of OsNPF4.5 was amplified and cloned into the Xenopus laevis oocyte expression vector pT7 Ts between the restriction sites Bgl II and Spe I, and then linearized with Xba I. Capped mRNA (cRNA) was synthesized in vitro using the Ambion mMessage mMachine kit (Ambion, AM1340). X. laevis oocytes were injected with 50 ng of OsNPF4.5 cRNA or 50 nL nuclease-free water. After injection, oocytes were cultured in ND-96 medium for 48 h and used for ¹⁵NO₃⁻-uptake assays. High- and low-affinity uptake assays in oocytes were conducted using 250 μM and 10 mM ¹⁵N—NaNO₃, respectively. Two-electrode voltage clamp assay was performed.

### Example. 2.19. ¹⁵N-Nitrate Uptake Activity In Vivo

Nitrate-uptake activity was determined using a ¹⁵N-labeling assay under hydroponic condition. Two-week-old seedlings of WT and transgenic plants were grown in IRRI nutrient solution containing 1 mM NH₄⁺ for 3 weeks and then deprived of N supply for 4 d. The N-starved plants were transferred to 0.1 mM CaSO4 solution for 1 min, and then resupplied with the nutrient solution containing either 2.5 mM ¹⁵NO₃⁻ or 2.5 mM ¹⁵NH₄⁺ for 10 min. The treated plants were transferred to 0.1 mM CaSO4 solution for 1 min before sampling. The ¹⁵N content in roots was determined with a DELTA V ADVANTAGE isotope Ratio MS as described above, and the uptake activity was calculated as the amount of ¹⁵N taken up per unit weight of roots per unit time.

### Example 2.20. Structural Alignment of Nitrate Transporters and Structure Modelling of OsNPF4.5

Multiple sequence alignment of NRT1.1 transporters and NPF4.5 orthologues were performed using MAFFT and secondary structures were assigned using ESPript 3.0. NCBI accession numbers used in the analyses are as follows: Brachypodium distachyon (Bd), XP_014754374.1; Zea mays (Zm), XP_020406064.1; Medicago truncatula (Mt), XP_024627880.1; Glycine max (Gc), XP_003532772.2; Vitis vinifera (Vv), XP_019078273.1; Populus euphratica (Pe), XP_011009674.1; Populus trichocarpa (Pt), XP_002305708.2; Helianthus annuus (Ha), XP_022013935.1; Solanum tuberosum (St), XP_006356126.1; Cannabis sativa (Cs), XP_030479547.1; Amborella trichopoda (At), XP_011624609.1. OsNPF4.5 structure was modelled using Rosseta (61) and visualized with PyMOL. Structure alignment between crystal structure of AtNRT1.1 and the model structure of OsNPF4.5 was analyzed using SuperPose server version 1.0 (62).

### Example 2.21. Statistical Analysis

The data were analyzed by ANOVA (SPSS 16.0; SPSS Inc., Chicago, Ill., USA) and Student's t test. Significance of differences was defined as *P<0.05, **P<0.01, ***P<0.001 or by different letters (P<0.05).

### Example 2.22. Accession Numbers

The sequence data from this Example can be found in The National Center for Biotechnology Information with the following accession numbers: OsNPF4.5 (LOC9271385), OsNPF6.4 (LOC9271131), OsPT11 (LOC4324187), OsHA1 (LOC4331281), OsNAR2.1 (LOC4329861), OsNRT2.1 (LOC4328051), OsNRT2.2 (LOC4328052), OsNPF1.3 (LOC4327022), OsNPF5.4 (LOC4348864), OsNPF7.2 (LOC4330372), OsNPF8.3 (LOC4336852), OsAMT3.1 (LOC107276856), OsNR1 (LOC4330867), OsNR2 (LOC4345798), OsGS1.1 (LOC4330649), MtNPF4.5 (LOC11406786), ZmNPF4.5 (LOC103652484), SbNPF4.5 (LOC8062188).

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A nitrate transporter gene OsNPF4.5, wherein the gene comprises the nucleotide sequence shown in SEQ ID NO:1 or a functional variant thereof, wherein the functional variant has at least 65% sequence identity to SEQ ID NO:1.

2. The nitrate transporter gene of claim 1, wherein the gene comprises the nucleotide sequence shown in SEQ ID NO:1.

3. The nitrate transporter gene of claim 1, wherein the gene comprises the functional variant of the nucleotide sequence shown in SEQ ID NO:1, and wherein the functional variant has at least 90% sequence identity to SEQ ID NO:1.

4. (canceled)

5. The nitrate transporter gene of claim 1, wherein the gene is in isolated and cDNA form, and wherein the gene is contained in a recombinant expression vector.

6. (canceled)

7. (canceled)

8. The nitrate transporter gene of claim 1, wherein the gene is contained in a plant as an exogenous gene.

9. The nitrate transporter gene of claim 8, wherein the plant is selected from the group consisting of rice, corn, soybean, cotton, tobacco, wheat, Medicago, maize, sorghum, and combinations thereof.

10. (canceled)

11. A nitrate transporter protein OsNPF4.5, wherein the protein comprises the amino acid sequence shown in SEQ ID NO: 2 or a functional variant thereof, wherein the functional variant has at least 65% sequence identity to SEQ ID NO:2.

12. The nitrate transporter protein of claim 11, wherein the protein comprises the amino acid sequence shown in SEQ ID NO:2.

13. The nitrate transporter protein of claim 11, wherein the protein comprises the functional variant of the amino acid sequence shown in SEQ ID NO:2, wherein the functional variant has at least 80% sequence identity to SEQ ID NO:2.

14. (canceled)

15. The nitrate transporter protein of claim 11, wherein the protein is in isolated form.

16. The nitrate transporter protein of claim 11, wherein the protein is contained in a plant as an exogenous protein.

17. The nitrate transporter protein of claim 16, wherein the plant is selected from the group consisting of rice, corn, soybean, cotton, tobacco, wheat, Medicago, maize, sorghum, and combinations thereof.

18. (canceled)

19. A method of enhancing plant growth, said method comprising:
introducing a nitrate transporter gene OsNPF4.5 into the plant, wherein the gene comprises the nucleotide sequence shown in SEQ ID NO:1 or a functional variant thereof, wherein the functional variant has at least 65% sequence identity to SEQ ID NO:1, and
wherein the introducing results in the expression of the nitrate transporter protein OsNPF4.5 in the plant, wherein the protein comprises the amino acid sequence shown in SEQ ID NO: 2 or a functional variant thereof, wherein the functional variant has at least 65% sequence identity to SEQ ID NO:2.

20. The method of claim 19, wherein the introduction occurs by a method selected from the group consisting of gene gun introduction methods, agrobacterium-mediated introduction methods, pollen tube channel introduction methods, or combinations thereof.

21. The method of claim 19, wherein the expressed nitrate transporter protein OsNPF4.5 enhances plant growth by enhancing the plant's absorption of nitrogen.

22. The method of claim 19, wherein the method enhances plant growth by at least 25%, at least 50%, or at least 100% relative to plants without the introduced nitrate transporter gene OsNPF4.5.

23. (canceled)

24. (canceled)

25. The method of claim 19, further comprising a step of associating the plant with an arbuscular mycorrhizal fungi, and wherein the associating comprises inoculating the root of the plant with the arbuscular mycorrhizal fungi.

26. (canceled)

27. The method of claim 19, wherein the plant is selected from the group consisting of rice, corn, soybean, cotton, tobacco, wheat, Medicago, maize, sorghum, and combinations thereof.

28. (canceled)

29. A genetically modified plant, wherein the genetically modified plant comprises an introduced nitrate transporter gene OsNPF4.5 wherein the gene comprises the nucleotide sequence shown in SEQ ID NO:1 or a functional variant thereof, wherein the functional variant has at least 65% sequence identity to SEQ ID NO:1.

30. The genetically modified plant of claim 29, wherein the plant is selected from the group consisting of rice, corn, soybean, cotton, tobacco, wheat, Medicago, maize, sorghum, and combinations thereof.

31. (canceled)

32. (canceled)
